(19) United States
(12) Patent Application Publication
(10) Pub. No.: US 2024/0052394 A1
(43) Pub. Date: Feb. 15, 2024

(54) BIOPRODUCTION OF ENANTIOPURE (R)- AND (S)-2-PHENYLGLYCINOL FROM STYRENE AND RENEWABLE FEEDSTOCKS VIA ARTIFICIAL ENZYME CASCADE

(71) Applicant: NATIONAL UNIVERSITY OF SINGAPORE, Singapore (SG)

(72) Inventors: Zhi Li, Singapore (SG); Balaji Sundara Sekar, Singapore (SG); Benedict Ryan Lukito, Singapore (SG)

(21) Appl. No.: 18/265,381

(22) PCT Filed: Dec. 6, 2021
(86) PCT No.: PCT/SG2021/050754

§ 371 Date: Jun. 5, 2023

(30) Foreign Application Priority Data
Dec. 7, 2020 (SG) 10202012190X

(51) Int. Cl. C12P 41/00; C12P 13/00; C12N 9/02; C12N 9/14; C12N 9/04; C12N 9/10; C12N 9/88

(52) U.S. Cl. CPC C12P 41/006; C12P 13/001; C12N 9/0071; C12Y 114/14011; C12N 9/14; C12Y 303/02003; C12N 9/0006; C12Y 101/01001; C12N 9/1096; C12N 9/88; C12Y 403/01024

(57) ABSTRACT

Disclosed herein are methods of bioproduction of enantiomerically pure or enantiomerically enriched 2-phenylglycinol or a derivative thereof by multiple enzyme-catalyzed chemical transformations in a one-pot reaction system.

### FIELD OF INVENTION

This invention relates to the bioproduction of useful and valuable enantiomerically pure or enantiomerically enriched 2-phenylglycinol or a derivative thereof. More particularly, the present invention provides methods of bioproduction of enantiomerically pure or enantiomerically enriched 2-phenylglycinol or a derivative thereof by one or more recombinant microbial cells genetically engineered to overexpress, relative to a wild type cell, at least one enzyme, which method comprises subjecting a starting material to a plurality of enzyme-catalyzed chemical transformations in a one-pot reaction system, wherein the starting material is selected from a group comprising glucose, L-phenylalanine or substituted L-phenylalanine, styrene or substituted styrene.

### BACKGROUND

The listing or discussion of a prior-published document in this specification should not necessarily be taken as an acknowledgement that the document is part of the state of the art or is common general knowledge.

Enantiopure β-amino alcohols are important chiral chemicals used as building blocks in the synthesis of several pharmaceutical compounds. Specifically, (R)-2-phenylglycinol 1a is used in the synthesis of novel antibiotic pyrazolopyrimidines, EGFR inhibitor thienopyrimidines, and 3-phosphoinositide-dependent protein kinase-1 (PDK1) inhibitor. (S)-2-phenylglycinol 1a can be used for the synthesis of (4S,5S)-2-phenyl-4-(methoxycarbonyl)-5-isopropyloxazoline, which is used for the synthesis of a neurotrophic agent. It is also used as a key precursor for the synthesis of chiral ligands for asymmetric annulation or cyclization, potent histone deacetylase inhibitors, and novel anti-cancer asymmetric triplex metallohelices. Due to its wide applications, enantiopure (R)- and (S)-2-phenylglycinol are high value compounds with the price of $40-$100 per kg, and a market demand of more than 1000 tons per year. Moreover, the substituted (R)-2-phenylglycinols 1b-Ik (FIG. 1a) are also useful pharmaceutical intermediates. For instance, (R)-2-amino-2-(4-methoxyphenyl)ethanol 1k and (1R,2R)-1-amino-2-indanol 11 are used in the synthesis of agonists and Alk5 inhibitors to treat cancers.

Several chemical methods were developed for the synthesis of (R) and (S)-1a. However, these methods have one or more disadvantages. For instance, methods for the reduction of D- or L-phenylglycine to (R)- or (S)-1a require expensive enantiopure substrates and utilize hazardous chemicals. In addition, the product yield in these methods is low with poor regio- and enantio-selectivity. Furthermore, kinetic resolution is required to obtain an enantiopure product but the kinetic resolution of racemic 1a to obtain enantiopure (R)- or (S)-1a suffers from low theoretical yield (50%, M. Periasamy, S. Sivakumar & M. N. Reddy, Synthesis 2003, 13, 1965-1967). The use of petroleum-derived chemicals, and energy-intensive multi-step process, further adds to the disadvantages. As the society is moving towards green and sustainable processes, unsustainable chemical-based methods are not preferred.

Enzyme catalysis is attractive due to high regio- and stereo-selectivity, mild reaction conditions, and environmental friendliness. The well-studied methods for the synthesis of chiral 2-phenylglycinol are asymmetric reduction of chiral amino ketones by ketone reductase, kinetic resolution of racemic amino alcohols by lipases, and amination of hydroxy ketones using transaminases. However, the enzymatic approaches developed for the synthesis of (R)- and
(S)-1a have drawbacks. They require special substrates and the substrates for these reactions are very expensive and commercially unavailable, which makes it difficult to scale up the process. In addition, the substrates are mainly derived from petroleum. Therefore, the process is unsustainable. Further, the kinetic resolution of racemic 1a suffers from low theoretical yield (50%, J .-D. Zhang et al., J. Biotechnol. 2019, 290, 24-32), thus making the process uneconomical.

Therefore, there is a need to develop green and sustainable methods for the production of enantiopure (R) and (S)-1a from cheap and renewable starting materials.

### SUMMARY OF INVENTION

Aspects and embodiments of the invention will now be discussed by reference to the following numbered clauses.
1. A method for producing an enantiomerically pure or enantiomerically enriched (R)- or (S)-2-phenylglycinol or a derivative thereof using one or more recombinant microbial cells genetically engineered to overexpress multiple enzymes, which method comprises subjecting styrene or a derivative thereof to multiple enzyme-catalyzed chemical transformations in a one-pot reaction system, optionally wherein at least one overexpressed enzyme is located on one or more plasmids or integrated in the chromosome of each of the one or more recombinant microbial cells.
2. The method of Clause 1, wherein the method produces enantiomerically pure or enantiomerically enriched 2-phenylglycinol or a derivative thereof, comprising the steps of:
   (a) generating phenylethanediol or a derivative thereof from styrene or a derivative thereof by conducting an epoxidation reaction catalyzed by an epoxidase to form styrene oxide or a derivative thereof and conducting a hydrolysis reaction catalyzed by an epoxide hydrolase on the epoxide, or by a dihydroxylation reaction catalyzed by a dioxygenase;
   (b) generating 2-hydroxyacetophenone or a derivative thereof from the phenylethanediol or a derivative thereof by an oxidation reaction catalyzed by an alcohol dehydrogenase or a secondary alcohol oxidase; and
   (c) generating enantiomerically pure or enantiomerically enriched 2-phenylglycinol or a derivative thereof from 2-hydroxyacetophenone or a derivative thereof by a transamination reaction catalyzed by a transaminase or a reductive amination reaction catalyzed by an amine dehydrogenase.
3. The method according to Clause 2, wherein:
   (a) the epoxidase is a styrene monooxygenase, optionally wherein the styrene monooxygenase is from Pseudomonas sp. VLB120, or its mutants or similar enzymes with more than 50% identity; and/or
   (b) the epoxide hydrolase, when used, is from Solanum tuberosum or its mutants or similar enzymes with more than 50% identity; and/or
   (c) the alcohol dehydrogenase, when used, is from Candida parapsilosis or, more preferably, Bacillus subtilis or their mutants or similar enzymes with more than 50% identity; and/or
   (d) the transaminase is from Bacillus megaterium, Chromobacterium violaceum, Vibrio fluvialis, Martelella mediterranea, Neosartora fischeri, Arthrobacter sp., or Aspergillus terreus or their mutants or similar enzymes with more than 50% identity.
4. The method according to and one of Clause 3, wherein
   (i) the epoxide hydrolase, when used, is selected from secondary alcohol dehydrogenase from Candida parapsilosis or, more preferably, (R,R)-butanediol dehydrogenase (BDHA) from Bacillus subtilis or its mutants or similar enzymes with more than 50% identity; and/or
   (ii) the transaminase is selected from Bacillus megaterium, Vibrio fluvialis, or Neosartora fischeri or their mutants or similar enzymes with more than 50% identity.
5. The method according to any one of the preceding clauses, wherein one of the one or more recombinant microbial cells genetically engineered to overexpress multiple enzymes (i.e. the one or more recombinant microbial cells genetically engineered to overexpress multiple enzymes) is selected from one or more of the group consisting of:
   (a) E. coli T7-pCDF-SMO-StEH, pET-BDHA-AlaDH, pRSF-BmTA;
   (b) E. coli T7-pCDF-SMO-StEH, pET-BmTA, pRSF-BDHA-AlaDH;
   (c) E. coli T7-pCDF-BDHA-AlaDH, pET-SMO-StEH, pRSF-BmTA;
   (d) E. coli T7-pCDF-BDHA-AlaDH, pET-BmTA, pRSF-SMO-StEH;
   (e) E. coli T7-pCDF-BmTA, pET-SMO-StEH, pRSF-BDHA-AlaDH; and
   (f) E. coli T7-pCDF-BmTA, pET-BDHA-AlaDH, pRSF-SMO-StEH.
6. The method according to Clause 5, wherein one of the one or more recombinant microbial cells genetically engineered to overexpress multiple enzymes (i.e. the one or more recombinant microbial cells genetically engineered to overexpress multiple enzymes) is E. coli T7-pCDF-SMO-StEH, pET-BDHA-AlaDH, pRSF-BmTA.
7. The method according to any one of the preceding clauses, wherein one of the one or more recombinant microbial cells genetically engineered to overexpress multiple enzymes (i.e. the one or more recombinant microbial cells genetically engineered to overexpress multiple enzymes) is selected from one or more of the group consisting of:
   (a) E. coli T7-pCDF-SMO-StEH, pET-BDHA-AlaDH, pRSF-NFTA;
   (b) E. coli T7-pCDF-SMO-StEH, pET-NFTA, pRSF-BDHA-AlaDH;
   (c) E. coli T7-pCDF-BDHA-AlaDH, pET-SMO-StEH, pRSF-NFTA;
   (d) E. coli T7-pCDF-BDHA-AlaDH, pET-NFTA, pRSF-SMO-StEH;
   (e) E. coli T7-pCDF-NFTA, pET-SMO-StEH, pRSF-BDHA-AlaDH; and
   (f) E. coli T7-pCDF-NFTA, pET-BDHA-AlaDH, pRSF-SMO-StEH.
8. The method according to any one of the preceding clauses, wherein one of the one or more recombinant microbial cells genetically engineered to overexpress multiple enzymes (i.e. the one or more recombinant microbial cells genetically engineered to overexpress multiple enzymes) is E. coli T7-pCDF-SMO-StEH, pET-NFTA, pRSF-BDHA-AlaDH.
9. The method according to any one of the preceding clauses, wherein the method further comprises providing styrene or a derivative thereof by generating trans-cinnamic acid or a derivative thereof from L-phenylalanine or a derivative thereof by a deamination reaction catalyzed by an ammonia lyase and generating styrene or a derivative thereof from the trans-cinnamic acid or a derivative thereof in a decarboxylation reaction catalyzed by a decarboxylase.
10. The method according to 9, wherein:
   (a) the ammonia lyase is phenylalanine ammonia lyase from Arabidopsis thaliana, or its mutants or similar enzymes with more than 50% identity; and/or
   (b) the decarboxylase is phenylacrylic acid decarboxylase from Aspergillus niger, or its mutants or similar enzymes with more than 50% identity.
11. The method according to any one of Clauses 1 to 6 and 9 to 10, wherein one of the one or more recombinant microbial cells genetically engineered to overexpress multiple enzymes (i.e. the one or more recombinant microbial cells genetically engineered to overexpress multiple enzymes) is E. coli T7-pACYC-PAL-PAD, pCDF-SMO-StEH, PET-BDHA-AlaDH, pRSF-BmTA.
12. The method according to any one of Clauses 1 to 4 and 7 to 10, wherein one of the one or more recombinant microbial cells genetically engineered to overexpress multiple enzymes (i.e. the one or more recombinant microbial cells genetically engineered to overexpress multiple enzymes) is E. coli T7-pACYC-PAL-PAD, pCDF-SMO-StEH, PET-BDHA-AlaDH, pRSF-NfTA.
13. The method according to any one of Clauses 9 to 12, wherein the method further 20 comprises cells that overexpress the natural L-phenylalanine biosynthetic pathway, which cells convert glucose or glycerol to L-phenylalanine.
14. The method according to Clause 13 wherein the microbial cells producing L-phenylalanine from glucose or glycerol overexpress at least one enzyme is selected from one or more of the group consisting of DAHP synthase (AroG), shikimate kinase (AroK), shikimate dehydrogenase (YdiB), chorismate mutase/prephenate dehydratase (PheA) and tyrosine aminotransferase (TyrB), or their mutants or similar enzymes with more than 50% identity, optionally wherein the microbial cells producing L-phenylalanine from glucose or glycerol overexpress at least one enzyme that is:
   (a) a feedback inhibition resistant mutant of AroG; and/or
   (b) a feedback inhibition resistant mutant of PheA.
15. The method according to Clause 14, wherein the microbial cells producing L-phenylalanine from glucose or glycerol overexpress at least one enzyme is one in which the microbial cells are mutated for deletion or inactivation of crr and/or tyrA genes.
16. The method according to any one of Clauses 13 to 15, wherein the microbial cells producing L-phenylalanine from glucose or glycerol that overexpress at least one enzyme is E. coli NST74-pACYC-PAL-PAD, pCDF-SMO-StEH, pET-BDHA-AlaDH, pRSF-BmTA or E. coli NST74-pACYC-PAL-PAD, pCDF-SMO-StEH, pET-BDHA-AlaDH, pRSF-NfTA.
17. The method according to any one of Clauses 13 to 15, wherein the microbial cells producing L-phenylalanine from glucose or glycerol that overexpress at least one enzyme is a combination of E. coli NST74-Phe with E. coli T7-pACYC-PAL-PAD, pCDF-SMO-StEH, pET-BDHA-AlaDH, pRSF-BmTA or is a combination of E. coli NST74-Phe with E. coli T7-pACYC-PAL-PAD, pCDF-SMO-StEH, pET-BDHA-AlaDH, pRSF-NfTA.
18. The method according to any one of the preceding clauses, wherein the one-pot reaction system comprises an aqueous medium.
19. The method according to any one of Clauses 1 to 17, wherein the one-pot reaction system comprises use of a bi-phasic medium, optionally wherein the bi-phasic medium consists of an aqueous and solid resin medium, or an aqueous and organic solvent medium (e.g. the organic solvent medium is selected from one or more alkanes solvents and/or one or more ester solvents).
20. The method according to any one of the preceding clauses, wherein the one-pot reaction system comprises use of absorbent resins or nanomaterials for in-situ removal of product.
21. An isolated nucleic acid molecule encoding at least one heterologous catalytic enzyme selected from the group comprising:
   (a) an epoxidase for conducting an epoxidation reaction to form styrene oxide or a derivative thereof from styrene or a derivative thereof and an epoxide hydrolase for conducting a hydrolysis reaction on the epoxide to provide phenylethanediol or a derivative thereof, or an oxygenase for generating phenylethanediol or a derivative thereof from styrene or a derivative thereof by a dihydroxylation reaction;
   (b) an alcohol dehydrogenase or a secondary alcohol oxidase for generating 2-hydroxyacetophenone or a derivative thereof from phenylethanediol by an oxidation reaction; and
   (c) a transaminase or an amine dehydrogenase for generating enantiomerically pure or enantiomerically enriched 2-phenylglycinol or a derivative thereof by a transamination reaction or a reductive amination from 2-hydroxyacetophenone or a derivative thereof; or
   (d) an ammonia lyase for generating trans-cinnamic acid or a derivative thereof from L-phenylalanine or a derivative thereof by a deamination reaction; and
   (e) a decarboxylase for generating styrene or a derivative thereof from trans-cinnamic acid or a derivative thereof in a decarboxylation reaction.
22. The isolated nucleic acid of clause 21, encoding a plurality of said catalytic enzymes.
23. The isolated nucleic acid molecule of clause 22, wherein said plurality of catalytic enzymes is arranged as at least one module selected from the group comprising:
   i) a module comprising heterologous nucleic acid sequences that, when expressed, enzymatically transforms styrene or a derivative thereof to phenylethanediol or a derivative thereof;
   ii) a module comprising heterologous nucleic acid sequences that, when expressed, enzymatically transforms phenylethanediol or a derivative thereof to a 2-hydroxyacetophenone or a derivative thereof;
   iii) a module comprising heterologous nucleic acid sequences that, when expressed, enzymatically transforms 2-hydroxyacetophenone or a derivative thereof to enantiomerically pure or enantiomerically enriched 2-phenylglycinol or a derivative thereof; and
   v) a module comprising heterologous nucleic acid sequences that, when expressed, enzymatically transforms an L-phenylalanine or a derivative thereof to styrene or a derivative thereof,
   or any combination thereof.
24. An expression construct comprising at least one nucleic acid molecule as described in any one of Clauses 21 to 23.
25. One or more recombinant prokaryotic or eukaryotic cells selected from the group comprising bacterial cells, yeast cells, mammalian cells and insect cells, wherein said cells comprise at least one expression construct as described in Clause 24.
26. The one or more recombinant prokaryotic or eukaryotic cells according to Clause 25, wherein said cells are recombinant bacterial cells.
27. The one or more recombinant prokaryotic or eukaryotic cells according to Clause 25 or Clause 26, wherein said enzymes have at least 50% amino acid identity with at least one enzyme selected from the group comprising:
   (a) a styrene monooxygenase from Pseudomonas sp. VLB120;
   (b) an epoxide hydrolase from Solanum tuberosum;
   (c) an alcohol dehydrogenase from Candida parapsilosis or, more preferably, Bacillus subtilis (e.g. the epoxide hydrolase is selected from secondary alcohol dehydrogenase from Candida parapsilosis or, more preferably, (R,R)-butanediol dehydrogenase (BDHA));
   (d) a transaminase from Bacillus megaterium, Chromobacterium violaceum, Vibrio fluvialis, Martelella mediterranea, Neosartora fischeri, Arthrobacter sp., or Aspergillus terreus (e.g. the transaminase is selected from Bacillus megaterium, Vibrio fluvialis, or Neosartora fischeri);
   (e) a phenylalanine ammonia lyase from Arabidopsis thaliana;
   (f) a phenylacrylic acid decarboxylase from Aspergillus niger.
28. The one or more recombinant cells of clause 27, wherein said cells are selected from:
   (a) E. coli T7-pCDF-SMO-StEH, pET-BDHA-AlaDH, pRSF-BmTA;
   (b) E. coli T7-pCDF-SMO-StEH, pET-BmTA, pRSF-BDHA-AlaDH;
   (c) E. coli T7-pCDF-BDHA-AlaDH, pET-SMO-StEH, pRSF-BmTA;
   (d) E. coli T7-pCDF-BDHA-AlaDH, pET-BmTA, pRSF-SMO-StEH;
   (e) E. coli T7-pCDF-BmTA, pET-SMO-StEH, pRSF-BDHA-AlaDH;
   (f) E. coli T7-pCDF-BmTA, PET-BDHA-AlaDH, pRSF-SMO-StEH.
   (g) E. coli T7-pCDF-SMO-StEH, pET-BDHA-AlaDH, pRSF-NFTA;
   (g) E. coli T7-pCDF-SMO-StEH, pET-NFTA, pRSF-BDHA-AlaDH;
   (i) E. coli T7-pCDF-BDHA-AlaDH, pET-SMO-StEH, pRSF-NFTA;
   (j) E. coli T7-pCDF-BDHA-AlaDH, pET-NFTA, pRSF-SMO-StEH;
   (k) E. coli T7-pCDF-NFTA, pET-SMO-StEH, pRSF-BDHA-AlaDH; and
   (I) E. coli T7-pCDF-NFTA, pET-BDHA-AlaDH, pRSF-SMO-StEH.
   (m) E. coli 17-pACYC-PAL-PAD, pCDF-SMO-StEH, pET-BDHA-AlaDH, pRSF-BmTA;
   (n) E. coli T7-pACYC-PAL-PAD, pCDF-SMO-StEH, pET-BDHA-AlaDH, pRSF-NfTA;
   (o) E. coli NST74-pACYC-PAL-PAD, pCDF-SMO-StEH, pET-BDHA-AlaDH, pRSF-BmTA; and
   (p) E. coli NST74-pACYC-PAL-PAD, pCDF-SMO-StEH, pET-BDHA-AlaDH, pRSF-NfTA.
29. The one or more recombinant cells of clause 28, wherein said cells are selected from:
   (i) E. coli T7-pCDF-SMO-StEH, pET-BDHA-AlaDH, pRSF-BmTA;
   (ii) E. coli T7-pCDF-SMO-StEH, pET-NFTA, pRSF-BDHA-AlaDH;
   (iii) E. coli T7-pACYC-PAL-PAD, pCDF-SMO-StEH, pET-BDHA-AlaDH, pRSF-BmTA; and
   (iv) E. coli T7-pACYC-PAL-PAD, pCDF-SMO-StEH, pET-BDHA-AlaDH, pRSF-NfTA.
30. A kit comprising at least one isolated nucleic acid according to any one of Clauses 21 to 24.

### DRAWINGS

FIG. 1 depicts the production of enantiopure (R)-2-phenylglycinols 1a-l and (S)-2-phenylglycinol 1a from styrenes 2a-k, indene 21, bio-based L-phenylalanine 6 and renewable feedstocks glucose 8 and glycerol 9. (a) Artificial enzyme cascade for the conversion of styrene 2a-k, indene 21 to (R)-1a-l and (S)-1a via one-pot epoxidation-hydrolysis-oxidation-amination reaction; (b) Artificial enzyme cascade for the biotransformation of L-phenylalanine 6 to (R)- and (S)-1a via one-pot deamination-decarboxylation-epoxidation-hydrolysis-oxidation-amination reaction; and (c) Conversion of glucose 8 and glycerol 9 to (R)- and (S)-1a via the combination of natural Shikimate pathway for the synthesis of 6 and the artificial enzyme cascade for the conversion of 6 to (R)- and (S)-1a. Enzymes involved are styrene monooxygenase (SMO), epoxide hydrolase (StEH), alcohol dehydrogenase (ADH), transaminase (TA), phenylalanine ammonia lyase (PAL) and phenylacrylic acid decarboxylase (PAD).

FIG. 2 depicts (a) production of hydroxyacetophenone 5a from 2 and 10 mM of (R)-phenylethanediol 4a by Escherichia coli (E. coli)- butanediol dehydrogenase (BDHA, 10 g cdw/L) or E. coli-secondary alcohol dehydrogenase (CpSADH, 10 g cdw/L); and (b) screening of transaminases for the conversion of 5a to (R)- or (S)-2-phenylglycinol 1a by biotransformation of 10 mM of 5a with E. coli strains expressing transaminases (20 g cdw/L) in phosphate buffer (200 mM, pH 8) containing glucose (0.5%) and amino donor (200 mM). Biotransformations were performed in duplicates with error bars showing ±SD.

FIG. 3 depicts a schematic representation of recombinant plasmids constructed for the cascade conversion of styrene 2a to (R)- and (S)-1a.

FIG. 4 depicts cascade biotransformation of styrene 2a to (R)- and (S)-1a using E. coli strains expressing the artificial enzyme cascade shown in FIG. 1a. (a) E. coli-SSBB-1-6 harboring three expression plasmids with different copy numbers for the expression of SMO, StEH, BDHA, alanine dehydrogenase (AlaDH) and transaminase from Bacillus megaterium (BmTA) at various expression levels for producing (R)-1a; (b) Sodium dodecyl sulphate-polyacrylamide gel electrophoresis (SDS-PAGE) analysis of crude extracts of E. coli-SSBB-1-6 showing the expression of enzymes for producing (R)-1a. Lane M, protein ladder; Lane 1-6, E. coli-SSBB-1-6; (c) Synthesis of (R)-1a from 10 mM of 2a by E. coli-SSBB-1-6 (20 g cdw/L) at 30° C. for 24 h; (d) E. coli-SSBN-1-6 harboring three expression plasmids with different copy numbers to express SMO, StEH, BDHA, AlaDH and transaminase from Neosartora fischeri (NfTA) at various expression levels for producing (S)-1a; (e) SDS-PAGE analysis of crude extracts of E. coli-SSBN-1-6 showing the expression of enzymes for producing (S)-1a. Lane M, protein ladder; Lane 7-12, E. coli-SSBN-1-6; and (f) Synthesis of (S)-1a from 5 mM of 2a by E. coli-SSBN-1-6 (20 g cdw/L) at 30° C. for 48 h. Reaction conditions: Two-phase system of 2 mL of phosphate buffer (200 mM, pH 8) and 2 mL of n-hexadecane containing 2a. Biotransformations were performed in duplicates with error bars showing ±SD.

FIG. 5 depicts cascade biotransformation of L-phenylalanine 6 to (R)- and (S)-1a using E. coli strains expressing the artificial enzyme cascade shown in FIG. 1b. (a) E. coli-PPSSBB and E. coli-PPSSBN harboring four expression plasmids for the expression of PAL, PAD, SMO, StEH, BDHA, AlaDH and BmTA or NfTA for the production of (R)- or (S)-1a, respectively; (b) SDS-PAGE analysis of crude extracts of E. coli-PPSSBB and E. coli-PPSSBN showing the expression of enzymes for producing (R)- or (S)-1a. Lane M, protein ladder; Lane C, E. coli T7 (negative control); Lane 13, E. coli-PPSSBB; Lane 14, E. coli-PPSSBN; and (c) Synthesis of (R)- and (S)-1a from 10 mM of 6 by E. coli-PPSSBB and E. coli- PPSSBN (15 g cdw/L), respectively in a two-phase system of phosphate buffer (5 mL, 200 mM, pH 8) containing 6 mL and 1 mL of n-hexadecane at 30° C. for 24 h. Biotransformations were performed in duplicates with error bars showing ±SD.

FIG. 6 depicts fermentation of glucose 8 or glycerol 9 to (R)- and (S)-1a using E. coli NST74(DE3) strains expressing the artificial enzyme cascade shown in FIG. 1c. (a) E. coli NST-PPSSBB and E. coli NST-PPSSBN harboring engineered Shikimate pathway and four expression plasmids for the expression of PAL, PAD, SMO, STEH, BDHA, AlaDH and BmTA or NfTA for the production of (R)- or (S)-1a, respectively; (b) SDS-PAGE analysis of crude extracts of E. coli NST-PPSSBB and E. coli NST-PPSSBN showing the expression of enzymes for the conversion of 6 to (R)- and (S)-1a. Lane M, protein ladder; Lane C, E. coli NST74(DE3); Lane 15, E. coli NST-PPSSBB; Lane 16, E. coli NST-PPSSBN; and (c) Synthesis of (R)- and (S)-1a from 20 g/L 8 or 9 by E. coli NST-PPSSBB and E. coli NST-PPSSBN, respectively via fermentation with 50 mL of modified M9 medium containing 8 or 9 and 10 mL of n-hexadecane at 25° C. for 24 h. Experiment was performed in duplicates with error bars showing ±SD.

FIG. 7 depicts (a) growing cells fermentation of glucose 8 or glycerol 9 using growing cells of E. coli NST-PPSSBB and E. coli NST-PPSSBN at 25° C. for 24 h for the production of (R)- and (S)-1a, respectively; and (b) coupled fermentation and biotransformation for the production of (R)- and (S)-1a from glucose 8 and glycerol 9. Fermentation was performed using growing cells of E. coli NST-Phe at 37° C. for 24-28 h for the production of 67-80 mM of 6 from 8 or 9. The fermented mixture containing biosynthesized 6 was used for the biotransformation of 6 to (R)- and (S)-1a at 30° C. for 24 h using E. coli-PPSSBB and E. coli-PPSSBN, respectively.

FIG. 8 depicts coupled fermentation and biotransformation for the conversion of glucose 8 and glycerol 9 to (R)- and (S)-1a from (a) E. coli NST-Phe harboring expression plasmid for the expression of key enzymes of Shikimate pathway for improved production of L-phenylalanine 6 was coupled with E. coli-PPSSBB and E. coli-PPSSBN harboring four expression plasmids for the expression of PAL, PAD, SMO, STEH, BDHA, AlaDH and BmTA or NfTA for the production of (R)- or (S)-1a, respectively from 8 and 9; and (b) synthesis of (R)- and (S)-1a by E. coli-PPSSBB and E. coli-PPSSBN, respectively from biosynthesized 6 produced by the fermentation of 8 and 9 by E. coli NST-Phe. Reaction procedure: Fermentation was performed using growing cells of E. coli NST-Phe at 37 ºC for 24-28 h for the production of 67-80 mM of 6 from 8 or 9. Biotransformation was performed by E. coli-PPSSBB or E. coli-PPSSBN (20 g cdw/L) at 30° C. for 24 h in a two-phase system containing 5 mL of fermented mixture containing biosynthesized 6 (10 mM), phosphate buffer (200 mM, pH 8) and n-hexadecane (1 mL). Experiment was performed in duplicates with error bars showing ±SD.

### DESCRIPTION

In a first aspect of the invention, there is provided a method for producing an enantiomerically pure or enantiomerically enriched (R)- or (S)-2-phenylglycinol or a derivative thereof using one or more recombinant microbial cells genetically engineered to overexpress multiple enzymes, which method comprises subjecting styrene or a derivative thereof to multiple enzyme-catalyzed chemical transformations in a one-pot reaction system. In embodiments of this aspect, at least one overexpressed enzyme is located on one or more plasmids or integrated in the chromosome of each of the one or more recombinant microbial cells.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

In embodiments of the invention, the styrene itself may be produced from L-phenylalanine by a further enzymatic cascade, with the L-phenylalanine being obtained either from the cell's natural metabolic processes or an enhanced enzymatic pathway that provides an up-regulated production of L-phenylalanine. These multiple reaction steps may be performed simultaneously or sequentially in one reaction vessel, to allow for the green, efficient, and economical production of enantiomerically pure or enantiomerically enriched (R)- or (S)-2-phenylglycinol or a derivative thereof directly from styrene or a derivative thereof, phenylalanine or a derivative thereof, glucose or glycerol. Such, one-pot cascade reactions may avoid the expensive and energy-consuming isolation and purification of intermediates, minimize waste generation, and overcome the possible thermodynamic hurdles normally encountered in traditional multi-step synthesis. For example, multiple enzymes may be co-expressed inside one recombinant microbe strain, and the whole cells of the strain may be directly applied as catalysts for a series of cascade reactions in one pot. Alternatively, the enzymes could be separately expressed in cells of different strains, purified individually, or immobilized (the purified enzymes or cells containing all or some of said enzymes). In any event, the biocatalysts (enzymes, cells, immobilized enzymes, and immobilized cells) can be mixed together in any suitable combination to effect the one pot transformation discussed herein.

As discussed hereinbefore, there is provided a method for producing an enantiomerically pure or enantiomerically enriched (R)- or (S)-2-phenylglycinol or a derivative thereof, which method comprises subjecting styrene or a derivative thereof (or other suitable starting materials mentioned herein) to multiple enzyme-catalyzed chemical transformations in a one-pot reaction system.

Styrene and derivatives thereof can be manufactured on very large scale in the petrochemical industry (e.g. by hydrocarbon cracking), and so form easily available and cheap starting materials for organic synthesis of the type discussed herein. For example, many aromatic and aliphatic alkenes are produced in very large amounts and at very low price. As discussed hereinbelow, styrenes and substituted styrenes are very useful substrates to produce various chiral aromatic 1,2-amino alcohols (such as phenylethanol amine, phenylglycinol, nor(pseudo)ephedrine) and α-amino acids (such as phenylglycine) in high enantiomeric purity.

While the use of suitable styrene and its derivatives generated from the petrochemical industry is envisaged, the current invention also allows for the conversion of L-phenylalanine and derivatives thereof into styrene and derivatives thereof by way of further enzyme-catalyzed transformations which will be discussed hereinbelow. This may enable access to styrene derivatives that may otherwise be difficult to obtain access to and provide a greater pool of possible styrene substrates for use in the enzyme-catalyzed transformations described herein.

It will be understood that the terms “enantiomerically pure” and “enantiomerically enriched” refer to enantiomers of a compound. “Enantiomers” refer to two stereoisomers of a compound which are non-superimposable mirror images of one another. Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes D and L or (+) and (−) are employed to designate the sign of rotation of plane-polarized light by the compound, with (−) or I (L) meaning that the compound is levorotatory. A compound prefixed with (+) or d (D) is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

When referred to herein, the term “enantiomerically enriched” may refer to an enantiomeric excess of 50% or more. For example, the methods disclosed herein may provide a final product having an enantiomeric excess of 60%, 70%, 80%, 90%, 95%, 98%, or 99% or more. In embodiments of the invention, only one enantiomer or diastereomer of a chiral compound is provided by the process described herein (i.e. the compound is “enantiomerically pure”).

When used herein, the term “derivative thereof” as applied to styrene and L-phenylalanine relates to a compound where the benzene ring contains one or more substituents (e.g. 1, 2, 3, 4 or 5, such as 1 to 3, such as 1 or 2 substituents) that are not H. Said substituents may be halo, alkyl, cycloalkyl, aryl, heterocyclic, OH, NH₂, SH and combinations thereof (e.g. alkyl aryl, Oalkyl, N(alkyl)H, N(alkyl)₂, N(alkyl)(aryl) etc).

Unless otherwise stated, the term “alkyl” refers to an unbranched or branched, saturated or unsaturated hydrocarbyl radical (so forming, for example, an alkenyl or alkynyl), which may be substituted or unsubstituted (with, for example, one or more halogen atoms). The alkyl group may be C₁₋₁₀ alkyl and, more preferably, C₁₋₆ alkyl (such as ethyl, propyl, (e.g. n-propyl or isopropyl), butyl (e.g. branched or unbranched butyl), pentyl or, more preferably, methyl). The terms “alkenyl” and “alkynyl” are to be interpreted accordingly.

Unless otherwise stated, the term “cycloalkyl” refers to an unbranched or branched, saturated or unsaturated hydrocarbyl radical (so forming, for example, a cycloalkenyl group) that may be substituted or unsubstituted. The cycloalkyl group may be C₃₋₁₂ cycloalkyl and, more preferably, C₆₋₁₀ (e.g. C₅₋₇) cycloalkyl. The term “cycloalkeny” is to be interpreted accordingly.

The term “halogen”, when used herein, includes fluorine, chlorine, bromine and iodine.

The term “aryl” when used herein includes C₆₋₁₄ (such as C₆₋₁₃ (e.g. C₆₋₁₀ )) aryl groups that may be substituted or unsubstituted. Such groups may be monocyclic, bicyclic or tricyclic and have between 6 and 14 ring carbon atoms, in which at least one ring is aromatic. The point of attachment of aryl groups may be via any atom of the ring system. However, when aryl groups are bicyclic or tricyclic, they are linked to the rest of the molecule via an aromatic ring. C₆₋₁₄ aryl groups include phenyl, naphthyl and the like, such as 1,2,3,4-tetrahydronaphthyl, indanyl, indenyl and fluorenyl. Most preferred aryl groups include phenyl.

When used herein, the term “aryl alkyl” is to be interpreted in line with the definitions provided hereinbefore for “alkyl” and “aryl”, where the point of attachment of the group to the rest of the compound of formula (I) or (II) is through the alkyl portion of the aryl alkyl group.

When used herein, the term “heterocyclic” refers to a fully saturated, partly unsaturated, wholly aromatic or partly aromatic ring system in which one or more (e.g. one to four) of the atoms in the ring system is other than carbon (i.e. a heteroatom, which heteroatom is preferably selected from N, O and S), and in which the total number of atoms in the ring system is between three and twelve (e.g. between five and ten). The heterocyclic groups may be substituted or unsubstituted. Heterocyclic groups that may be mentioned include 7-azabicyclo[2.2.1]heptanyl, 6-azabicyclo[3.1.1]heptanyl, 6-azabicyclo[3.2.1]octanyl, 8-azabicyclo[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo[3.2.1]octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thirranyl, thiolanyl, thiomorpholinyl, trithianyl (including 1,3,5-trithianyi), tropanyl, benzothiadiazolyl (including 2,1,3-benzothiadiazolyl), isothiochromanyl and, more preferably, acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro- 2H-1 ,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1 ,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heterocyclic groups may, where appropriate, be located on any atom in the ring system including a heteroatom. The point of attachment of heterocyclic groups may be via any atom in the ring system including (where appropriate) a heteroatom (such as a nitrogen atom), or an atom on any fused carbocyclic ring that may be present as part of the ring system. Heterocyclic groups may also be in the N- or S- oxidised form. Heterocyclic groups that may be mentioned herein include cyclic amino groups such as pyrrolidinyl, piperidyl, piperazinyl, morpholinyl or a cyclic ether such as tetrahydrofuranyl.

When used herein, the term “heterocyclic alkyl” is to be interpreted in line with the definitions provided hereinbefore for “alkyl” and “heterocyclic”, where the point of attachment of the group to the rest of the compound of formula (I) or (II) is through the alkyl portion of the heterocyclic alkyl group.

The substituents mentioned herein may be substituted or unsubstituted. When the substituents are substituted, they may be substituted with one or more of the groups selected from the group of halogen (e.g., a single halogen atom or multiple halogen atoms forming, in the latter case, groups such as CF₃ or an alkyl group bearing Cl₃), cyano, nitro, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR₆R_{c}, OC(═O)Rₐ, OC(═O)NRR_{c}, NR_{b}C(═O)ORₑ, NRₐC(═O)NR_{b}R_{c}, NR_{d}S(═O)₂NR_{b}R_{c}, NRₐP(═O)₂NR_{b}R_{c}, NR_{b}C(═O)Rₐ, or NR_{b}P(═O)₂Rₑ, wherein Rₐ is hydrogen, alkyl, cycloalkyl, alkenyl, cycloalkenyl, alkynyl, heterocycle, or aryl; R_{b}, R_{c} and Rₐ are independently hydrogen, alkyl, cycloalkyl, heterocycle, aryl, or said R_{b} and R_{c} together with the N to which they are bonded optionally form a heterocycle; and Rₑ is alkyl, cycloalkyl, alkenyl, cycloalkenyl, alkynyl, heterocycle, or aryl. It will be appreciated that these substituted groups may be unsubstituted or are themselves substituted with one or more halogen atoms.

For the avoidance of doubt, in cases in which the identity of two or more may be the same, the actual identities of the respective substituents are not in any way interdependent unless otherwise specified.

Specific styrene and L-phenylalanine derivatives that may be mentioned herein include those in which the phenyl ring of styrene and/or phenylalanine is mono- or disubstituted by (a) substituent(s) selected from F, Cl, Br, CH₃ or OCH₃. For example, the phenyl ring of styrene and/or phenylalanine may be monosubstituted by o-F, m-F, p-F, m-Cl, p-Cl, m-Br, p-Br, m-CH₃, p-CH₃, or p-OCH₃.

In particular embodiments of the invention that may also be mentioned herein, the styrene derivative may be indene which is either unsubstituted or substituted as described for styrene above.

The terms “amino acid” or “amino acid sequence,” as used herein, refer to an oligopeptide, peptide, polypeptide, or protein sequence, or a fragment of any of these, and to naturally occurring or synthetic molecules. Where “amino acid sequence” is recited herein to refer to an amino acid sequence of a naturally occurring protein molecule, “amino acid sequence” and like terms are not meant to limit the amino acid sequence to the complete native amino acid sequence associated with the recited protein molecule.

The term “isolated” is herein defined as a biological component (such as a nucleic acid, peptide or protein) that has been substantially separated, produced apart from, or purified away from other biological components in the cell of the organism in which the component naturally occurs, i.e., other chromosomal and extra-chromosomal DNA and RNA, and proteins. Nucleic acids, peptides and proteins which have been isolated thus include nucleic acids and proteins purified by standard purification methods. The term also embraces nucleic acids, peptides and proteins prepared by recombinant expression in a host cell as well as chemically synthesized nucleic acids.

The phrases “nucleic acid” or “nucleic acid sequence,” as used herein, refer to an oligonucleotide, nucleotide, polynucleotide, or any fragment thereof, to DNA or RNA of genomic or synthetic origin which may be single-stranded or double-stranded and may represent the sense or the antisense strand, to peptide nucleic acid (PNA), or to any DNA-like or RNA-like material. In the context of the invention, “fragments” refers to those nucleic acid sequences which are greater than about 60 nucleotides in length, and most preferably are at least about 100 nucleotides, at least about 1000 nucleotides, or at least about 10,000 nucleotides in length which are not full-length native sequence but retain catalytic enzyme activity.

The term “oligonucleotide,” as used herein, refers to a nucleic acid sequence of at least about 6 nucleotides to 60 nucleotides, preferably about 15 to 30 nucleotides, and most preferably about 20 to 25 nucleotides, which can be used in PCR amplification or in a hybridization assay or microarray. As used herein, the term “oligonucleotide” is substantially equivalent to the terms “amplimers,” “primers,” “oligomers,” and “probes,” as these terms are commonly defined in the art.

The terms ‘variant’ and ‘mutant’ are used interchangeably herein. The at least one nucleic acids encoding at least one catalytic enzyme may encode a variant or mutant of the exemplified catalytic enzyme which retains activity. A “variant” of a catalytic enzyme, as used herein, refers to an amino acid sequence that is altered by one or more amino acids. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing catalytic activity may be found using computer programs well known in the art, for example, DNASTAR software. In some embodiments, variant enzymes are at least 60%, 65%, 70%, 75%, 80%, 85%, 90%, 95%, 96%, 97%, 98%, 99%, or more, homologous or identical at the amino acid level to an exemplary amino acid sequence described herein (e.g., catalase, alcohol dehydrogenase, α-transaminase) or a functional fragment thereof—e.g., over a length of about: 50, 55, 60, 65, 70, 75, 80, 85, 90, 95, or 100% of the length of the mature reference sequence.

The terms ‘phenylacetaldehyde reductase’ (PAR) and ‘alcohol dehydrogenase’ (ADH), as referred to herein, are used interchangeably.

A vector can include one or more catalytic enzyme nucleic acid(s) in a form suitable for expression of the nucleic acid(s) in a host cell. Preferably the recombinant expression vector includes one or more regulatory sequences operatively linked to the nucleic acid sequence(s) to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences such as the T7 IPTG-inducible promoters disclosed in the Examples herein. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., catalytic enzyme proteins, fusion proteins, and the like).

The recombinant expression vectors of the invention can be designed for expression of catalytic enzyme proteins in prokaryotic or eukaryotic cells. For example, polypeptides of the invention can be expressed in bacteria (e.g., E. coli), insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector(s) can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. Gene 1988, 67: 31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

To maximize recombinant protein expression in E. coli is to express the protein in a host bacterium with an impaired capacity to proteolytically cleave the recombinant protein (Gottesman, S. Gene Expression Technology: Methods in Enzymology 1990 185, Academic Press, San Diego, Calif. 119-128). Another strategy is to alter the nucleic acid sequence of the nucleic acid to be inserted into an expression vector so that the individual codons for each amino acid are those preferentially utilized in E. coli (Wada et al., Nucleic Acids Res. 1992 20: 2111-2118). Such alteration of nucleic acid sequences of the invention can be carried out by standard DNA synthesis techniques and is described in the Examples.

The catalytic enzyme expression vector can be a yeast expression vector, a vector for expression in insect cells, e.g., a baculovirus expression vector, a vector for expression in bacterial cells, e.g. a plasmid vector, or a vector suitable for expression in mammalian cells.

When used in mammalian cells, the expression vector's control functions can be provided by viral regulatory elements. For example, commonly used promoters are derived from polyoma, Adenovirus 2, cytomegalovirus and Simian Virus 40.

In a preferred embodiment, the promoter is an inducible promoter, e.g., a promoter regulated by a steroid hormone, by a polypeptide hormone (e.g., by means of a signal transduction pathway), by a chemical (e.g., Isopropyl β-D-1-thiogalactopyranoside (IPTG)) or by a heterologous polypeptide.

The methods described herein make use of enzymes to catalyse a sequence of reactions. While these reactions may be performed individually or, more particularly, two or more of them in combination, all of the reactions may be combined into a cascade reaction sequence that provides the product from the initial starting material in one pot, thereby eliminating the need for isolation of the intermediates and, potentially, increasing the overall yield of the reaction sequence. These cascade reactions may involve the use of one or more reactive components selected from the group consisting of cells, immobilized cells, cell extracts, isolated enzymes and immobilized enzymes in said reaction vessel.

Another aspect the invention provides at least one expression construct comprising at least one nucleic acid sequence that is heterologous according to any aspect of the invention. Preferably the at least one construct comprises a plasmid suitable for expression of at least one catalytic enzyme in a bacterium.

Another aspect the invention provides a host cell which includes at least one nucleic acid molecule described herein, e.g., at least one catalytic enzyme nucleic acid molecule within a recombinant expression vector or a catalytic enzyme nucleic acid molecule containing sequences which allow homologous recombination into a specific site of the host cell's genome. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, at least one catalytic enzyme protein can be expressed in bacterial cells (such as E. coli), insect cells (such as

Spodoptera frugiperda Sf9 cells), yeast or mammalian cells (such as Chinese hamster ovary cells (CHO) or COS cells (African green monkey kidney cells CV-1 origin SV40 cells; Gluzman Cell 1981, 123: 175-182)). Other suitable host cells are known to those skilled in the art.

One or more vector DNAs can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. For example, according to the invention a host cell may comprise one, two, three, four or more plasmids, each of which may express at least one catalytic enzyme directed to a chemical transformation in the pathway from an alkene starting material to an enantiomerically pure or enantiomerically enriched 1,2-amino alcohol or a-amino acid. The host cell may also include a vector to express catalytic enzymes for providing the alkene by, for example, generating a vinyl carboxylic acid from an a-amino acid by a deamination reaction catalyzed by an ammonia lyase and generating the alkene from the vinyl carboxylic acid in a decarboxylation reaction catalyzed by a decarboxylase.

In a preferred embodiment, the catalytic enzymes required to transform an alkene starting material to an enantiomerically pure or enantiomerically enriched 1,2-amino alcohol or α-amino acid are arranged on expression vectors as modules, wherein each module comprises a combination of catalytic enzymes to perform specific reactions within the overall system. For example, a first module may comprise enzymes for the dihydroxylation of a terminal alkene to a 1,2-diol co-expressed on a plasmid; a second module may comprise enzymes for the oxidation-amination of a 1,2-diol to a 1,2-amino alcohol co-expressed on a plasmid; a third module may comprise enzymes for the double oxidation of a 1,2-diol to an α-hydroxy acid co-expressed on a plasmid; a fourth module may comprise enzymes for the oxidation-amination of α-hydroxy acid to α-amino acid co-expressed on a plasmid; and a fifth module may comprise enzymes for the deamination-decarboxylation of an α-amino acid to an alkene. Arrangements of such enzymes as modules allow flexibility in constructing a serial cascade of reactions in one pot. One or more modules may be engineered onto the same plasmid. For example, a host cell comprising the said first and third modules, on the same or separate plasmids, is capable of catalyzing the conversion of a terminal alkene to a 1,2-amino alcohol. Likewise a host cell comprising said first, a second and fourth module is capable of catalyzing the conversion of a terminal alkene to an α-amino acid. If module 5 is co-expressed in the host cell, an α-amino acid feed stock can be provided for the cell to generate its own terminal alkene for the cascade reactions.

In one aspect of the invention there is provided one or more recombinant prokaryotic or eukaryotic cells selected from the group comprising bacterial cells, yeast cells, mammalian cells and insect cells, wherein said cells comprise at least one expression construct and/or heterologous nucleic acid molecule that encodes at least one catalytic enzyme required in the pathway from alkene to enantiomerically pure or enantiomerically enriched 1,2-amino alcohol or from an α-amino acid to enantiomerically pure or enantiomerically enriched 1,2-amino alcohol.

The cells may contain a single expression vector or construct, such as a plasmid, which expresses a single catalytic enzyme or co-expresses a plurality of catalytic enzymes as described herein under the control of at least one regulatory element. The catalytic enzymes may be arranged in the plasmid as an individual artificial operon under the control of a promoter with one ribosome-binding site before every gene, or arranged with individual promoters. Accordingly, a one-pot synthesis cascade may be achieved with cells expressing all required catalytic enzymes on one or several plasmids, or with different recombinant cells which each express a specific repertoire of catalytic enzymes, providing the necessary cells are included for a particular chemical transformation.

In another preferred embodiment, said catalytic enzymes for use in the invention have at least 50%, 60%, 65%, 70%, 75%, 80%, 85%, 90%, 95%, 96%, 97%, 98%, 99%, or more, homology or amino acid identity with at least one enzyme mentioned herein.

A host cell of the invention can be used to produce (i.e., express) one or more catalytic enzyme proteins. Accordingly, the invention further provides methods for producing one or more catalytic enzyme proteins, e.g., one or more catalytic enzyme proteins described herein, using the host cells of the invention. In one embodiment, the method includes culturing the host cell of the invention (into which one or more recombinant expression vector(s) encoding one or more catalytic enzyme proteins has/have been introduced) in a suitable medium such that one or more catalytic enzyme proteins is/are produced. In another embodiment, the method further includes isolating one or more catalytic enzyme proteins from the medium or the host cell.

According to another aspect of the invention there is provided a kit comprising at least one recombinant cell, expression construct or isolated nucleic acid according to any aspect of the invention. Preferably, the kit can be used to provide one or more components for a one-pot system to enzyme-catalyze the production of an enantiomerically pure or enantiomerically enriched (R)- or (S)-2-phenylglycinol or a derivative thereof from a suitable starting material.

The method in the first aspect of the invention that produces enantiomerically pure or enantiomerically enriched 2-phenylglycinol or a derivative thereof may comprise the steps of:
(a) generating phenylethane diol or a derivative thereof from styrene or a derivative thereof by conducting an epoxidation reaction catalyzed by an epoxidase to form styrene oxide or a derivative thereof and conducting a hydrolysis reaction catalyzed by an epoxide hydrolase on the epoxide, or by a dihydroxylation reaction catalyzed by a dioxygenase;
(b) generating 2-hydroxyacetophenone or a derivative thereof from the phenylethane diol or a derivative thereof by an oxidation reaction catalyzed by an alcohol dehydrogenase or a secondary alcohol oxidase; and
(c) generating enantiomerically pure or enantiomerically enriched 2-phenylglycinol or a derivative thereof from 2-hydroxyacetophenone or a derivative thereof by a transamination reaction catalyzed by a transaminase or a reductive amination reaction catalyzed by an amine dehydrogenase.

In the above enzymatic cascade, one or more of the following may apply:
(a) the epoxidase may be a styrene monooxygenase. For example, the styrene monooxygenase may be from Pseudomonas sp. VLB120, or its mutants or similar enzymes with more than 50% identity;
(b) the epoxide hydrolase, when used, may be from Solanum tuberosum or its mutants or similar enzymes with more than 50% identity;
(c) the alcohol dehydrogenase, when used, may be from Candida parapsilosis or, more preferably, Bacillus subtilis or their mutants or similar enzymes with more than 50% identity; and
(d) the transaminase may be from Bacillus megaterium, Chromobacterium violaceum, Vibrio fluvialis, Martelella mediterranea, Neosartora fischeri, Arthrobacter sp., or Aspergillus terreus or their mutants or similar enzymes with more than 50% identity.

Yet more particularly, the epoxide hydrolase, when used, may be selected from secondary alcohol dehydrogenase from Candida parapsilosis or, more preferably, (R,R)-butanediol dehydrogenase (BDHA) from Bacillus subtilis or its mutants or similar enzymes with more than 50% identity and/or the transaminase may be selected from Bacillus megaterium, Vibrio fluvialis, or Neosartora fischeri or their mutants or similar enzymes with more than 50% identity.

As noted above, the currently disclosed invention may make use of one or more recombinant microbial cells genetically engineered to overexpress multiple enzymes. Thus, in embodiments of the invention that may be mentioned herein, the one of the one or more recombinant microbial cells genetically engineered to overexpress multiple enzymes (i.e. the one or more recombinant microbial cells genetically engineered to overexpress multiple enzymes) may be selected from one or more of the group consisting of:
(a) E. coli T7-pCDF-SMO-StEH, pET-BDHA-AlaDH, pRSF-BmTA;
(b) E. coli T7-pCDF-SMO-StEH, pET-BmTA, pRSF-BDHA-AlaDH;
(c) E. coli T7-pCDF-BDHA-AlaDH, pET-SMO-StEH, pRSF-BmTA;
(d) E. coli T7-pCDF-BDHA-AlaDH, pET-BmTA, pRSF-SMO-StEH;
(e) E. coli T7-pCDF-BmTA, pET-SMO-StEH, pRSF-BDHA-AlaDH; and
(f) E. coli T7-pCDF-BmTA, pET-BDHA-AlaDH, pRSF-SMO-StEH.

In particular embodiments of the invention, the one of the one or more recombinant microbial cells genetically engineered to overexpress multiple enzymes (i.e. the one or more recombinant microbial cells genetically engineered to overexpress multiple enzymes) may be E. coli T7-pCDF-SMO-StEH, pET-BDHA-AlaDH, pRSF-BmTA.

In further embodiments of the invention that may be mentioned herein, the one of the one or more recombinant microbial cells genetically engineered to overexpress multiple enzymes (i.e. the one or more recombinant microbial cells genetically engineered to overexpress multiple enzymes) may be selected from one or more of the group consisting of:
(a) E. coli T7-pCDF-SMO-StEH, pET-BDHA-AlaDH, pRSF-NFTA;
(b) E. coli T7-pCDF-SMO-StEH, pET-NFTA, pRSF-BDHA-AlaDH;
(c) E. coli T7-pCDF-BDHA-AlaDH, pET-SMO-StEH, pRSF-NFTA;
(d) E. coli T7-pCDF-BDHA-AlaDH, pET-NFTA, pRSF-SMO-StEH;
(e) E. coli T7-pCDF-NFTA, pET-SMO-StEH, pRSF-BDHA-AlaDH; and
(f) E. coli T7-pCDF-NFTA, pET-BDHA-AlaDH, pRSF-SMO-StEH.

In particular embodiments of the invention, the one of the one or more recombinant microbial cells genetically engineered to overexpress multiple enzymes (i.e. the one or more recombinant microbial cells genetically engineered to overexpress multiple enzymes) may be E. coli T7-pCDF-SMO-StEH, pET-NFTA, pRSF-BDHA-AlaDH.

As noted hereinbefore, the method may make use of other starting materials other than styrene or its derivatives. Thus, in further embodiments of the invention, the method may further comprise providing styrene or a derivative thereof by generating trans-cinnamic acid or a derivative thereof from L-phenylalanine or a derivative thereof by a deamination reaction catalyzed by an ammonia lyase and generating styrene or a derivative thereof from the trans-cinnamic acid or a derivative thereof in a decarboxylation reaction catalyzed by a decarboxylase.

In the conversion of L-phenylalanine or a derivative thereof to styrene or a derivative thereof, one or more of the following enzymes may be used:
(a) the ammonia lyase may be phenylalanine ammonia lyase from Arabidopsis thaliana, or its mutants or similar enzymes with more than 50% identity; and
(b) the decarboxylase may be phenylacrylic acid decarboxylase from Aspergillus niger, or its mutants or similar enzymes with more than 50% identity.

In particular embodiments of the invention, the conversion of L-phenylalanine or a derivative thereof to enantiomerically pure or enantiomerically enriched (R)- or (S)-2-phenylglycinol or a derivative thereof may make use of one of the one or more recombinant microbial cells genetically engineered to overexpress multiple enzymes (i.e. the one or more recombinant microbial cells genetically engineered to overexpress multiple enzymes), said one or more recombinant microbial cells may be E. coli T7-pACYC-PAL-PAD, pCDF-SMO-StEH, pET-BDHA-AlaDH, pRSF-BmTA.

In additional or alternative embodiments of the invention, the conversion of L-phenylalanine or a derivative thereof to enantiomerically pure or enantiomerically enriched (R)- or (S)-2-phenylglycinol or a derivative thereof may make use of one of the one or more recombinant microbial cells genetically engineered to overexpress multiple enzymes (i.e. the one or more recombinant microbial cells genetically engineered to overexpress multiple enzymes), said one or more recombinant microbial cells may be E. coli T7-pACYC-PAL-PAD, pCDF-SMO-StEH, pET-BDHA-AlaDH, pRSF-NfTA.

As noted above, the method disclosed herein may make use of the cellular apparatus to generate the feedstock for the enzymatic cascade from glucose or glycerol. Thus, in embodiments of the invention, the method may further comprise cells that overexpress the natural L-phenylalanine biosynthetic pathway, which cells convert glucose or glycerol to L-phenylalanine. For example, the microbial cells producing L-phenylalanine from glucose or glycerol may overexpress at least one enzyme that is selected from one or more of the group consisting of DAHP synthase (AroG), shikimate kinase (AroK), shikimate dehydrogenase (YdiB), chorismate mutase/prephenate dehydratase (PheA) and tyrosine aminotransferase (TyrB), or their mutants or similar enzymes with more than 50% identity. More particularly, the microbial cells producing L-phenylalanine from glucose or glycerol may overexpress at least one enzyme that is:
(a) a feedback inhibition resistant mutant of AroG;
(b) a feedback inhibition resistant mutant of PheA.

As will be appreciated, one or both of these enzymes may be overexpressed.

In particular embodiments of the invention, the microbial cells producing L-phenylalanine from glucose or glycerol overexpress at least one enzyme may be one in which the microbial cells are mutated for deletion or inactivation of crr and/or tyrA genes.

In particular embodiments of the invention, the microbial cells producing L-phenylalanine from glucose or glycerol that overexpress at least one enzyme may be E. coli NST74-pACYC-PAL-PAD, pCDF-SMO-StEH, pET-BDHA-AlaDH, pRSF-BmTA or E. coli NST74-pACYC-PAL-PAD, pCDF-SMO-StEH, pET-BDHA-AlaDH, pRSF-NfTA. Additionally or alternatively, the microbial cells producing L-phenylalanine from glucose or glycerol that overexpress at least one enzyme may be a combination of E. coli NST74-Phe with E. coli T7-pACYC-PAL-PAD, pCDF-SMO-StEH, pET-BDHA-AlaDH, pRSF-BmTA or may be a combination of E. coli NST74-Phe with E. coli T7-pACYC-PAL-PAD, pCDF-SMO-StEH, pET-BDHA-AlaDH, pRSF-NfTA.

The method may make use of any suitable medium to conduct the reaction. For example, the one-pot reaction system may comprise an aqueous medium. Alternatively or additionally, the one-pot reaction system may comprise use of a bi-phasic medium, optionally wherein the bi-phasic medium consists of an aqueous and solid resin medium, or an aqueous and organic solvent medium (e.g. the organic solvent medium is selected from one or more alkanes solvents and/or one or more ester solvents). Alkane solvents that may be mentioned herein include, but are not limited to n-hexadecane.

In embodiments of the invention disclosed herein, the one-pot reaction system may comprise use of absorbent resins or nanomaterials for in-situ removal of product.

In a further aspect of the invention, there is provided an isolated nucleic acid molecule encoding at least one heterologous catalytic enzyme selected from the group comprising:
(a) an epoxidase for conducting an epoxidation reaction to form styrene oxide or a derivative thereof from styrene or a derivative thereof and an epoxide hydrolase for conducting a hydrolysis reaction on the epoxide to provide phenylethane diol or a derivative thereof, or an oxygenase for generating phenylethane diol or a derivative thereof from styrene or a derivative thereof by a dihydroxylation reaction;
(b) an alcohol dehydrogenase or a secondary alcohol oxidase for generating 2-hydroxyacetophenone or a derivative thereof from phenylethane diol by an oxidation reaction; and
(c) a transaminase or an amine dehydrogenase for generating enantiomerically pure or enantiomerically enriched 2-phenylglycinol or a derivative thereof by a transamination reaction or a reductive amination from 2-hydroxyacetophenone or a derivative thereof; or
(d) an ammonia lyase for generating trans-cinnamic acid or a derivative thereof from L-phenylalanine or a derivative thereof by a deamination reaction; and
(e) a decarboxylase for generating styrene or a derivative thereof from trans-cinnamic acid or a derivative thereof in a decarboxylation reaction.

As will be appreciated, the isolated nucleic acid molecule above may encode for two or more, such as three or more, such as four or more, or all of said catalytic enzymes. That is, the isolated nucleic acid may encode a plurality of said catalytic enzymes. In such embodiments, said plurality of catalytic enzymes may be arranged as at least one module selected from the group comprising:
i) a module comprising heterologous nucleic acid sequences that, when expressed, enzymatically transforms styrene or a derivative thereof to phenylethane diol or a derivative thereof;
ii) a module comprising heterologous nucleic acid sequences that, when expressed, enzymatically transforms phenylethane diol or a derivative thereof to a 2-hydroxyacetophenone or a derivative thereof;
iii) a module comprising heterologous nucleic acid sequences that, when expressed, enzymatically transforms 2-hydroxyacetophenone or a derivative thereof to enantiomerically pure or enantiomerically enriched 2-phenylglycinol or a derivative thereof; and
v) a module comprising heterologous nucleic acid sequences that, when expressed, enzymatically transforms an L-phenylalanine or a derivative thereof to styrene or a derivative thereof,
or any combination thereof.

A further aspect of the invention may relate to an expression construct comprising at least one nucleic acid molecule as described hereinbefore. The expression construct may comprise one or or more recombinant prokaryotic or eukaryotic cells selected from the group comprising bacterial cells, yeast cells, mammalian cells and insect cells. For example, the one or more recombinant prokaryotic or eukaryotic cells may be one in which said enzymes (encoded by the at least one nucleic acid molecule) have at least 50% amino acid identity with at least one enzyme selected from the group comprising:
(a) a styrene monooxygenase from Pseudomonas sp. VLB120;
(b) an epoxide hydrolase from Solanum tuberosum;
(c) an alcohol dehydrogenase from Candida parapsilosis or, more preferably, Bacillus subtilis (e.g. the epoxide hydrolase is selected from secondary alcohol dehydrogenase from Candida parapsilosis or, more preferably, (R,R)-butanediol dehydrogenase (BDHA));
(d) a transaminase from Bacillus megaterium, Chromobacterium violaceum, Vibrio fluvialis, Martelella mediterranea, Neosartora fischeri, Arthrobacter sp., or Aspergillus terreus (e.g. the transaminase is selected from Bacillus megaterium, Vibrio fluvialis, or Neosartora fischeri);
(e) a phenylalanine ammonia lyase from Arabidopsis thaliana;
(f) a phenylacrylic acid decarboxylase from Aspergillus niger.

In particular embodiments of the invention, the one or more recombinant cells may be selected from:
(a) E. coli T7-pCDF-SMO-StEH, pET-BDHA-AlaDH, pRSF-BmTA;
(b) E. coli T7-pCDF-SMO-StEH, pET-BmTA, pRSF-BDHA-AlaDH;
(c) E. coli T7-pCDF-BDHA-AlaDH, pET-SMO-StEH, pRSF-BmTA;
(d) E. coli T7-pCDF-BDHA-AlaDH, pET-BmTA, pRSF-SMO-StEH;
(e) E. coli T7-pCDF-BmTA, pET-SMO-StEH, pRSF-BDHA-AlaDH;
(f) E. coli T7-pCDF-BmTA, PET-BDHA-AlaDH, pRSF-SMO-StEH.
(g) E. coli T7-pCDF-SMO-StEH, pET-BDHA-AlaDH, pRSF-NFTA;
(g) E. coli T7-pCDF-SMO-StEH, pET-NFTA, pRSF-BDHA-AlaDH;
(i) E. coli T7-pCDF-BDHA-AlaDH, pET-SMO-StEH, pRSF-NFTA;
(j) E. coli T7-pCDF-BDHA-AlaDH, pET-NFTA, pRSF-SMO-StEH;
(k) E. coli T7-pCDF-NFTA, pET-SMO-StEH, pRSF-BDHA-AlaDH; and
(I) E. coli T7-pCDF-NFTA, PET-BDHA-AlaDH, pRSF-SMO-StEH.
(m) E. coli 17-pACYC-PAL-PAD, pCDF-SMO-StEH, pET-BDHA-AlaDH, pRSF-BmTA;
(n) E. coli T7-pACYC-PAL-PAD, pCDF-SMO-StEH, pET-BDHA-AlaDH, pRSF-NfTA;
(o) E. coli NST74-pACYC-PAL-PAD, pCDF-SMO-StEH, PET-BDHA-AlaDH, pRSF-BmTA; and
(p) E. coli NST74-pACYC-PAL-PAD, pCDF-SMO-StEH, pET-BDHA-AlaDH, pRSF-NfTA.

In more particular embodiments that may be mentioned herein, the one or more recombinant cells may be selected from:
(i) E. coli T7-pCDF-SMO-StEH, pET-BDHA-AlaDH, pRSF-BmTA;
(ii) E. coli T7-pCDF-SMO-StEH, pET-NFTA, pRSF-BDHA-AlaDH;
(iii) E. coli T7-pACYC-PAL-PAD, pCDF-SMO-StEH, pET-BDHA-AlaDH, pRSF-BmTA; and
(iv) E. coli T7-pACYC-PAL-PAD, pCDF-SMO-StEH, pET-BDHA-AlaDH, pRSF-NfTA.

In a further aspect of the invention, there is provided a kit comprising at least one isolated nucleic acid as described hereinbefore.

Further aspects and embodiments of the invention will now be discussed by reference to the following non-limiting examples.

### EXAMPLES

### Materials

The following chemicals were purchased from Sigma-Aldrich: L-Phenylalanine 6 (99%), D-glucose 8 (99%), glycerol 9 (99%), (R)-phenylglycinol 1a (99%), (S)-phenylglycinol 1a (99%), (R)-phenylethanediol 4a (99%), 2-hydroxyacetophenone 5a (99%), styrene 2a (99%), 2-fluorostyrene 2b (98%), indene 2l (98%), kanamycin sulfate (95%), ampicillin sodium salt (96%), streptomycin sulfate (95%), ammonium persulfate (≥98%), sodium citrate (≥98%), thiamine HCl (98%), ferrous sulfate (99%), ammonium hydroxide (28%, NH₃ in H₂O), NaCl (99.5%), betaine (99%), n-hexadecane (99%), ethyl oleate (99%), KH₂PO₄ (99%), trifluoroacetic acid (TFA, 99%), sulphuric acid (≥99%), acetophenone (99%), Na2HPO4.2H₂O (99%), NH₄Cl(99%), NaOH (99%), Na₂SO₄ (99%), benzyl alcohol (99%), MgSO₄ (99%), MnSO₄ (99%), CaCl₂ (99%), FeCl₃ (99%), CoCl₂·6H₂O (99%), CuCl₂·2H₂O (99%), ZnCl₂·4H₂O (98%), NaMoO₄ (99%), H₃BO₄ (9%), HCl (9%), NaBH₄ (>99%), 2-amino-2-(3-bromophenyl)acetic acid (>99%), iodine (12, >99%), KOH (>99%), methanol (MeOH, >99%), tetrahydrofuran (THF, >99%), ethyl acetate (>99%), glucose (>99%), glycerol (>99%), dichloromethane (CH₂Cl₂, >99%), D-Alanine (>98%), L-Glutamate (>99%), L-Alanine (>99%) and isopropylamine (>99%).

The following chemicals were obtained from other suppliers: 3-fluorostyrene 2c (>97%), 4-fluorostyrene 2d (>98%), 3-chlorostyrene 2e (>98%), 4-chlorostyrene 2f (>98%), 3-bromostyrene 2g (>97%), 4-bromostyrene 2h (>95%), 3-methylstyrene 2i (>97%), 4-methylstyrene 2j (>96%), 4-methoxystyrene 2k (>98%), (1R,2R)-1-amino-2-indanol 1l were purchased from TCI chemicals. (R)-2-amino-2-(2-fluorophenyl)ethanol 1b (97%), 2-amino-2-(3-fluorophenyl)ethanol 1c (97%), 2-amino-2-(4-fluorophenyl)ethanol 1d (97%), (R)-2-amino-2-(4-fluorophenyl)ethanol 1d (97%), 2-amino-2-(3-chlorophenyl)ethanol 1e (97%), (S)-2-amino-2-(3-chlorophenyl)ethanol 1e (97%), 2-amino-2-(4-chlorophenyl)ethanol 1f (97%), (S)-2-amino-2-(3-bromophenyl)ethanol 1g (97%), 2-amino-2-(4-bromophenyl)ethanol 1h (97%), 2-amino-2-(3-methylphenyl)ethanol 1i (97%), 2-amino-2-(4-methylphenyl)ethanol 1j (97%), (S)-2-amino-2-(4-methylphenyl)ethanol 1j (97%) and 2-amino-2-(4-methoxyphenyl)ethanol 1k (97%) were purchased from Acrotein ChemBio Inc. Isopropyl β-D-1-thiogalactopyranoside (IPTG, 99%) was purchased from 1^{st} base Singapore. Ethyl acetate (HPLC grade) and acetonitrile (HPLC grade) were obtained from Tedia. Luria broth (LB, Miller) powder and BactoTM yeast extract were purchased from Becton Dickinson Germany.

The plasmid isolation kit and gel extraction kit were purchased from Omega Bio-tek, Inc. USA. All DNA-modifying enzymes including DNA polymerases and restriction digestion enzymes required in gene cloning were purchased from Thermo Fisher Scientific USA. SDS sample loading dye and SDS-PAGE gels were purchased from Genscript Singapore.

### Analytical Techniques

### High-Performance Liquid Chromatography (HPLC) Analysis

The samples were centrifuged at 30,000× g for 10 min to separate the cells, aqueous and organic phase. The products were present only in the aqueous phase and analyzed by reverse phase HPLC using a Shimadzu prominence HPLC system. The HPLC was equipped with Agilent Poroshell 120 SB-C18 column (150×4.6 mm, 2.7 μm) and maintained at 25° C. A mobile phase of 80% water with 0.1% TFA, and 20% acetonitrile was used at a flow rate of 0.5 mL/min. The products were detected using photodiode array detector measuring UV absorbance at 210 nm.

Retention time: 4a, 4.33 min; 5a, 6.02 min; 6, 3.55 min; 1a, 4.22 min; 1b, 4.25 min; 1c, 4.62 min; 1d, 4.71 min; 1e, 6.12 min; 1f, 6.71 min; 1g, 6.93 min; 1h, 7.74 min; 1i, 5.62 min; 1j, 6.20 min; 1k, 4.78 min; and 1l, 4.32 min.

Analysis of ee Values with HPLC

The ee values of the products were analyzed using reverse phase HPLC system equipped with Daicel Crownpak CR (+) (150×4.0 mm, 5 μm) column and maintained at 25° C. A mobile phase of water with 0.1% TFA was used at a flow rate of 1.0 mL/min. The ee values of products 1h and 1k, were analyzed as aforementioned but with modified mobile phase: 90% water with 0.1% TFA and 10% methanol (MeOH) was used at a flow rate of 1.0 mL/min. The products were detected using photodiode array detector measuring UV absorbance at 210 nm.

Retention time: (R)-1a, 4.62 min; (S)-1a, 4.19 min; (R)-1b, 4.19 min; (R)-1c, 5.81 min; (R)-1d, 6.03 min; (R)-1e, 19.78 min; (R)-1f, 18.74 min; (R)-1g, 4.66 min; (R)-1h, 13.61 min; (R)-1i, 13.48 min; (R)-1j, 12.81 min; (R)-1k, 4.53 min; and (R)-1l, 5.84 min.

### SDS-PAGE Analysis

The harvested cell pellets were resuspended in 20 mM Tris-HCI buffer (pH 8.0) to give a cell concentration of 10 OD₆₀₀. 1 mL of the cells was added with 1 g of 0.1 mm glass beads in a 2 mL lysis tube, followed by homogenization using FastPrep-24. The lysate was centrifuged at 2000× g for 5 min to remove whole cells and glass beads. 50 μL of the clear lysate was mixed with 10 μL of 5× SDS sample loading dye, heated at 95° C. for 15 min, and resolved in 12% SDS-PAGE gel. The molecular weights of the expressed proteins were predicted from the DNA sequence using ExPASy Compute pl/Mw tool. Predicted molecular weights: AlaDH, 48.6 kDa; BDHA, 37.3 kDa; BmTA, 53 kDa; NfTA, 36 kDa; PAD, 55.2 kDa; PAL, 77.9 kDa; SMO, 46.4 kDa; StEH, 36.2 kDa.

### Polymerase Chain Reaction (PCR)

PCR was performed using the Thermal cycler purchased from Biorad USA. PCR experiments were performed using Phusion polymerase from Thermo Scientific USA. The protocols from the manufacturers were followed for PCR amplifications.

### NMR Spectroscopy

The NMR was performed using the NMR spectrometer purchased from Bruker USA. The samples were prepared in CD₃OD. ¹H NMR and ¹³C NMR were performed at 400 MHz and 100 MHz, respectively.

### Synthesis of Racemic 2-Amino-2-(3-Bromophenyl)Ethanol 1g

Racemic 2-amino-2-(3-bromophenyl)ethanol 1g was synthesized in our lab according to a reported method (WO/2014/106606).

A solution of racemic 2-amino-2-(3-bromophenyl)acetic acid (23 mg), and NaBH4 (9 mg) in anhydrous THF (2 mL) were added dropwise into a solution of iodine (26 mg) in anhydrous THF (2 mL). The reaction mixture was stirred at 70° C. for 20 h, followed by quenching with MeOH at room temperature. The reaction mixture was treated with 20% aqueous KOH and stirred overnight. The crude 1 g was obtained by extraction with ethyl acetate, followed by evaporation. The extracted compound was further purified by preparative thin layer chromatography (TLC) with CH₂Cl₂:MeOH:NH₃ (28% aqueous solution) of 100:10:1 as the mobile phase (R_{f}˜0.3). The purified compound was checked by HPLC (>95% purity) and 1H NMR.

¹H NMR (400 MHZ, CD₃OD): δ7.57 (s, 1H), 7.41 (d, J=7.82 Hz, 1H), 7.33 (d, J=7.74 Hz, 1H), 7.25 (t, J=7.78 Hz, 1H), 4.84 (s, 3H), 3.92 (dd, J=7.82 5.08 Hz, 1H), 3.66 (dd, J=4.97 10.81 Hz, 1H), 3.54 (dd, J=7.44 10.81 Hz, 1H).

### General Procedure for the Preparation of Medium

### Medium for Genetic Engineering, Seed Culturing and Strain Maintenance

LB medium—Tryptone (10 g/L), Yeast extract (5 g/L) and NaCl (10 g/L).

Medium for Culturing E. coli Strains for Whole-Cell Biotransformation

Modified M9 medium—Na₂HPO₄ (6 g/L), KH₂PO₄ (3 g/L), NH₄Cl (1 g/L), NaCl (0.5 g/L), MgSO₄ (1 mM), CaCl₂ (0.1 mM), glucose (20 g/L), yeast extract (6 g/L), and trace metal solution (1 mL). Trace metal solution: CoCl₂·2H₂O (0.1 g/L), H₃BO₃ (0.1 g/L), Na₂MoO₄ (0.1 g/L), FeCl₃ (8.3 g/L), ZnCl₂ (0.84 g/L), CuCl₂·2H₂O (0.13 g/L) and MnCl₂ (0.016 g/L).

Medium for the Production of L-Phenylalanine by E. coli NST-Phe

Fermentation medium—Glucose or glycerol (10 g/L), KH₂PO₄ (5 g/L), (NH4)₂SO₄ (10 g/L), MgSO₄ (5 g/L), yeast extract (5 g/L), betaine (1 g/L), FeSO₄ (15 mg/L), and MnSO₄ (15 mg/L). Glucose or glycerol was maintained at 10 g/L by feeding 500 g/L of solution, and the pH was maintained at ˜6.8 with NH₄OH solution (30%).

### General Procedure for Recombinant Strain Construction

The duet vectors such as pACYCDuet, pETDuet, pCDFDuet and pRSFDuet were used as expression plasmids. The insert DNA was either PCR amplified or synthesized as double stranded DNA, and ligated with appropriate expression plasmid using restriction-digestion cloning method. The ligated DNA fragments were transformed in E. coli DH5α competent cells using heat-shock method. The colonies were verified using colony PCR and the positive clones were sequenced. The positive clones were cultured in 5 mL of LB medium for 12 h at 37° C. and the expression plasmids were extracted. The isolated plasmids were transformed into E. coli T7 express to obtain recombinant E. coli strains for enzyme expression.

### General Procedure for the Cultivation of E. coli Strains

The glycerol stock of recombinant E. coli was inoculated in 2 mL of LB medium with appropriate antibiotics (50 μg/mL kanamycin, 25 μg/mL chloramphenicol, 50 μg/mL streptomycin, and 100 μg/mL ampicillin), and grown at 37° C. and 220 rpm for 6-8 h. 1 mL of the culture was added to 50 mL of modified M9 medium with appropriate antibiotics (50 μg/mL kanamycin, 25 μg/mL chloramphenicol, 50 μg/mL streptomycin, and 100 μg/mL ampicillin) in a 250 mL flask, and grown at 37° C. and 220 rpm. Antibiotics were reduced to 50% for strains containing more than two plasmids. At 2 h, 0.5 mM of IPTG was added and the growth temperature was set at 22° C. After 16 h of growth at 22° C. , the cells were harvested by centrifugation at 2680× g for 10 min. The cell pellets were resuspended in appropriate volume of phosphate buffer and used for biotransformation.

### Example 1. Recombinant strain construction

DNA sequences of recombinant genes
>BDHA (SEQ ID. NO: 1)
>CpSADH (SEQ ID. NO: 2)
>HnTA (SEQ ID. NO: 3)
>DpgTA (SEQ ID. NO: 4)
>EeaTA (SEQ ID. NO: 5)
>VITA (SEQ ID. NO: 6)
>CvTA (SEQ ID. NO: 7)
>mVITA (SEQ ID. NO: 8). Mutation of VITA: Y150F and V153A.
>BmTA (SEQ ID. NO: 9)
>MmTA (SEQ ID. NO: 10)
>NITA (SEQ ID. NO: 11)
>ArRTA (SEQ ID. NO: 12)
>AtTA (SEQ ID. NO: 13)
   E. coli Strain Expressing Enzymes for the Cascade Conversion of Styrene 2a to (R)- and (S)-2-Phenylglycinol 1a

The genes coding for BDHA from Bacillus subtilis BGSC1A1, CpSADH from Candida parapsilosis and 11 transaminases were synthesized as gBlock fragments and amplified by PCR. BdhA and CpSADH genes were cloned in pETDuet and the transaminases genes were cloned in pRSFDuet. The genes coding for enzymes in the cascade (SMO, StEH, BdhA, AlaDH and BmTA or NfTA) were cloned into three expression plasmids (pETDuet, pCDFDuet and pRSFDuet) with different copy numbers and antibiotic resistance. The expression plasmids were transformed into E. coli T7 to obtain 12 different recombinant strains (E. coli-SSBB-1-6 and E. coli-SSBN-1-6) with varying expression levels of the enzymes in the artificial cascade (Table 1).

TABLE 1
List of E. coli strains used in this study for
the production of (R)- and (S)-2-phenylglycinol 1a.

| Strain | Recombinant plasmids | Reaction |
|---|---|---|
| E. coli-BDHA | E. coli T7-pETDuet-BDHA | 4 to 5 |
| E. coli-CpSADH | E. coli T7-pETDuet-CpSADH | 4 to 5 |
| E. coli-HnTA | E. coli T7-pRSFDuet-HnTA | 5 to 1 |
| E. coli-DpgTA | E. coli T7-pRSFDuet-DpgTA | 5 to 1 |
| E. coli-EeaTA | E. coli T7-pRSFDuet-EeaTA | 5 to 1 |
| E. coli-VfTA | E. coli T7-pRSFDuet-VfTA | 5 to 1 |
| E. coli-CvTA | E. coli T7-pRSFDuet-CvTA | 5 to 1 |
| E. coli-mVfTA | E. coli T7-pRSFDuet-mVfTA | 5 to 1 |
| E. coli-BmTA | E. coli T7-pRSFDuet-BmTA | 5 to 1 |
| E. coli-MmTA | E. coli T7-pRSFDuet-MmTA | 5 to 1 |
| E. coli-NfTA | E. coli T7-pRSFDuet-NfTA | 5 to 1 |
| E. coli-ArRTA | E. coli T7-pRSFDuet-ArRTA | 5 to 1 |
| E. coli-AtTA | E. coli T7-pRSFDuet-AtTA | 5 to 1 |
| E. coli-SSBB-1 | E. coli T7-pCDF-SMO-StEH, pET-BDHA- | 2 to (R)-1 |
| | AlaDH, pRSF-BmTA | |
| E. coli-SSBB-2 | E. coli T7-pCDF-SMO-StEH, pET-BmTA, pRSF- | 2 to (R)-1 |
| | BDHA- AlaDH | |
| E. coli-SSBB-3 | E. coli T7-pCDF-BDHA- AlaDH, pET-SMO- | 2 to (R)-1 |
| | StEH, pRSF-BmTA | |
| E. coli-SSBB-4 | E. coli T7-pCDF-BDHA- AlaDH, pET-BmTA, | 2 to (R)-1 |
| | pRSF-SMO-StEH | |
| E. coli-SSBB-5 | E. coli T7-pCDF-BmTA, pET-SMO-StEH, pRSF- | 2 to (R)-1 |
| | BDHA- AlaDH | |
| E. coli-SSBB-6 | E. coli T7-pCDF-BmTA, pET-BDHA- AlaDH, | 2 to (R)-1 |
| | pRSF-SMO-StEH | |
| E. coli-SSBN-7 | E. coli T7-pCDF-SMO-StEH, pET-BDHA- | 2 to (S)-1 |
| | AlaDH, pRSF-NFTA | |
| E. coli-SSBN-8 | E. coli T7-pCDF-SMO-StEH, pET-NFTA, pRSF- | 2 to (S)-1 |
| | BDHA- AlaDH | |
| E. coli-SSBN-9 | E. coli T7-pCDF-BDHA- AlaDH, pET-SMO- | 2 to (S)-1 |
| | StEH, pRSF-NFTA | |
| E. coli-SSBN-10 | E. coli T7-pCDF-BDHA- AlaDH, pET-NFTA, | 2 to (S)-1 |
| | pRSF-SMO-StEH | |
| E. coli-SSBN-11 | E. coli T7-pCDF-NFTA, pET-SMO-StEH, pRSF- | 2 to (S)-1 |
| | BDHA- AlaDH | |
| E. coli-SSBN-12 | E. coli T7-pCDF-NFTA, pET-BDHA- AlaDH, | 2 to (S)-1 |
| | pRSF-SMO-StEH | |
| E. coli-PPSSBB | E. coli T7-pACYC-PAL-PAD, pCDF-SMO-StEH, | 6 to (R)-1 |
| | pET-BDHA- AlaDH, pRSF-BmTA | |
| E. coli-PPSSBN | E. coli T7-pACYC-PAL-PAD, pCDF-SMO-StEH, | 6 to (S)-1 |
| | pET-BDHA- AlaDH, pRSF-NfTA | |
| E. coli NST-PPSSBB | E. coli NST74(DE3)-pACYC-PAL-PAD, pCDF- | 8 or 9 to (R)-1 |
| | SMO-StEH, pET-BDHA- AlaDH, pRSF-BmTA | |
| E. coli NST-PPSSBN | E. coli NST74(DE3)-pACYC-PAL-PAD, pCDF- | 8 or 9 to (S)-1 |
| | SMO-StEH, pET-BDHA- AlaDH, pRSF-NfTA | |
| E. coli NST-Phe | E. coli NST74(DE3)-pCDF-AroG*-YdiB-AroK- | 8 or 9 to 6 |
| | PheA*-TyrB | |

E. coli Strain Expressing Enzymes for the Cascade Conversion of L-phenylalanine 6 to (R)- and (S)-2-Phenylglycinol 1a

E. coli-SSBB-1 and E. coli-SSBN-2 were further engineered for the cascade conversion of L-5 phenylalanine 6 to phenylglycinol 1a. pACYCDuet expressing PAL and PAD from our previous study (Y. Zhou, S. Wu & Z. Li, Adv. Synth. Catal. 2017, 359, 4305-4316) was transformed into E. coli-SSBB-1 (pCDF-SMO-StEH, pET-BDHA-AlaDH, pRSF-NFTA) and E. coli-SSBN-2 (pCDF-SMO-StEH, pET-NFTA, pRSF-BDHA-AlaDH) to obtain E. coli-PPSSBB (produce (R)-1a) and E. coli-PPSSBN (produce (S)-1a), respectively.

E. coli Strain Expressing Enzymes for the Production of (R)- and (S)-2-phenylglycinol 1a from Glucose 8 and Glycerol 9

For the synthesis of phenylglycinol 1a from glucose 8 and glycerol 9, E. coli NST74 (DE3) was used as the expression strain, which was previously engineered to produce L-phenylalanine (Y. Zhou et al., ChemSusChem 2018, 11, 2221-2228). pCDF-SMO-StEH, pET-BdhA-AlaDH, pRSF-BmTA/NfTA and pACYC-PAL-PAD were transformed into E. coli NST74 (DE3) to obtain E. coli NST-PPSSBB (produce (R)-1a) and E. coli NST-PPSSBN (produce (S)-1a), respectively.

### Example 2. Design of Cascade Biotransformation for the Production of (R)- and (S)-2-Phenylglycinol 1a from Styrene 2a

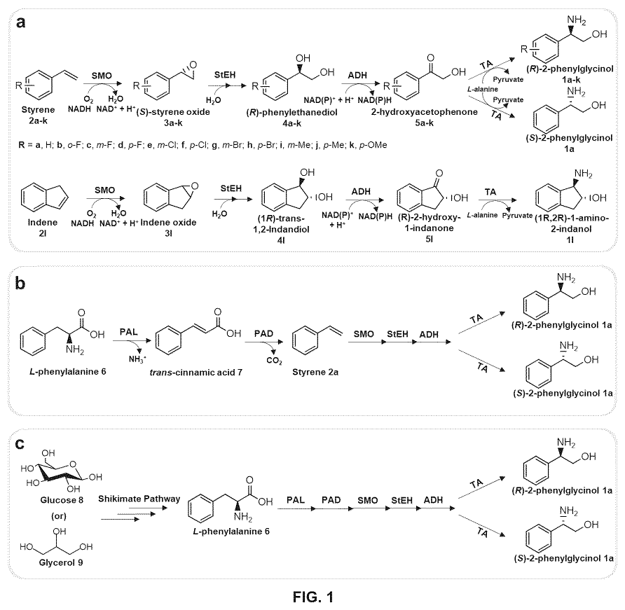

To convert styrene 2a to (R)- and (S)-2-phenylglycinol 1a, we designed artificial cascade with four enzymes for the epoxidation-hydrolysis-oxidation-transamination reactions (FIG. 1a).

Conversion of (R)-phenylethanediol 4a to hydroxyacetophenone 5a

Biotransformation was performed at 30° C. with 5 mL suspension of E. coli-BDHA or E. coli-CpSADH (10 g cdw/L) in phosphate buffer (200 mM, pH 8) containing 2 or 10 mM of 4a. At 12 h, 50 μL of the aqueous sample was collected and analyzed in reverse-phase HPLC for the production of 5a.

### Whole Cell Biotransformation for Hydroxyacetophenone 5a to (R)- and (S)-Phenylglycinol 1a

Whole cell biotransformation was performed at 30° C. for 24 h with 5 mL suspension of E. coli strains expressing transaminases (20 g cdw/L) in potassium phosphate buffer (KP, 200 mM, pH 8) containing 10 mM of 5a. Glucose (0.5%) and 200 mM of an amino donor selected from D-Alanine, Glutamate, L-Alanine and isopropylamine, were added to the reaction mixture at 0 h. Then, additional glucose (0.5%) and amino donor (100 mM) were added at 20 h. After 24 h reaction, the samples were analysed by reverse phase HPLC to determine the concentration and ee of 1a. Biotransformations were performed in triplicates and the mean values were presented.

### Results and Discussion

The conversion of styrene 2a to (R)-phenylethanediol 4a by epoxidation-hydrolysis reaction were achieved by SMO from Pseudomonas sp. and StEH from Solanum tuberosum (S. Wu, J. Liu & Z. Li, ACS Catal. 2017, 7, 5225-5233; Y. Zhou, S. Wu & Z. Li, Angew. Chem. 2016, 128, 11819-11822; and S. Wu et al., ACS Catal. 2014, 4, 409-420). Conversion of 4a to 2-hydroxyacetophenone 5a by oxidation reaction could be achieved by the use of alcohol dehydrogenase. Amination of 5a to (R)- and (S)-2-phenylglycinol 1a, respectively, could be achieved by using (R) and (S)-enantioselective transaminases.

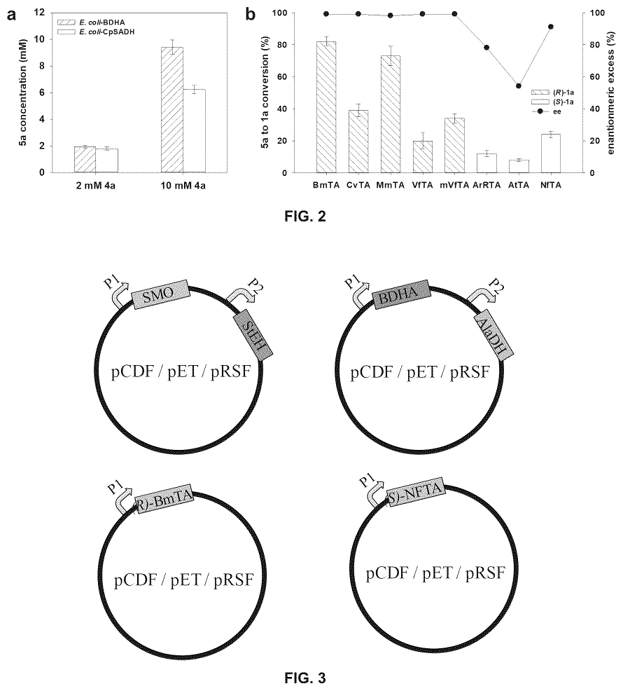

For the oxidation of (R)-phenylethanediol 4a to 2-hydroxyacetophenone 5a, BDHA from Bacillus subtilis BGSC1A1 and CpSADH from Candida parapsilosis were examined. These enzymes were reported for the efficient production of hydroxy ketones (S. Wu, Z. Li, ChemCatChem 2018, 10, 2164-2178; J. Zhang et al., ACS Catal. 2015, 5, 51-58; and J. Liu & Z. Li, Biotechnol. Bioeng. 2019, 116, 536-542). The genes coding for BDHA and CpSADH were cloned in individual pET28a plasmid under the control of T7 promoter, and the two recombinant plasmids were transformed into separate E. coli T7 strains to obtain E. coli-BDHA expressing BDHA and E. coli-CpSADH expressing CpSADH, respectively. The resting cells of E. coli-BDHA and E. coli-CpSADH (10 g cdw/L) were evaluated for oxidation of 2 mM of 4a, giving 95-100% conversion to 5a in both cases (FIG. 2a). The concentration of 4a was increased to 10 mM, and the strains were evaluated. E. coli-BDHA efficiently produced 9.4 mM of 5a with 94% conversion within 12 h, whereas E. coli-CpSADH produced only 6.24 mM of 5a with 62% conversion at the same time. Therefore, BDHA was chosen as the enzyme for the enzyme cascade for the oxidation of 4a in the cascade biotransformations.

Transaminase is the key enzyme in this cascade catalytic reaction for the production of enantiopure 2-phenylglycinol 1a. For the conversion of 5a to (R)-1a, databases were searched for (S)-enantioselective transaminases with substrate preference similar to 5a and simple amino donors such as glutamate, L-alanine and isopropylamine. Eight (S)-enantioselective transaminases such as HnTA, DpgTA, EeaTA, VITA, CvTA, mVITA (A. Nobili et al., ChemCatChem 2015, 7, 757-760), BmTA and MmTA, were identified with substrate preference similar to 5a. The genes coding for the eight (S)-enantioselective transaminases were cloned in individual pRSFDuet-1 plasmid and transformed into E. coli T7 strains, each expressing one of the eight different transaminases (Tables 1-2). 10 g cdw/L of resting cells of E. coli strains expressing eight transaminases were used for the biotransformation of 10 mM of 5a in KP buffer containing glucose (0.5%) and 200 mM of amino donor (D-, L-alanine or glutamate) for 24 h.

TABLE 2
Screening of transaminases for the conversion of hydroxyacetophenone
5a to (R)- and (S)-Phenylglycinol 1a.

| | Source of | | | Conv. | e.e. |
|---|---|---|---|---|---|
| Strain | transaminase | Amino donor | Product | (%) | (%) |
| E. coli-HnTA | Hyphomonas neptunium | D-Alanine | — | — | — |
| E. coli-DpgTA | Pseudomonas stutzeri | Glutamate | — | — | — |
| E. coli-EeaTA | Escherichia coli | Glutamate | — | — | — |
| E. coli-VfTA | Vibrio fluvialis | L-Alanine | (R)-1 | 20 | >99 |
| E. coli-CvTA | Chromobacterium violaceum | L-Alanine | (R)-1 | 39 | >99 |
| E. coli-mVfTA | Vibrio fluvialis | L-Alanine | (R)-1 | 34 | >99 |
| E. coli-BmTA | Bacillus megaterium | L-Alanine | (R)-1 | 82 | >99 |
| E. coli-MmTA | Martelella mediterranea | L-Alanine | (R)-1 | 73 | 98 |
| E. coli-NfTA | Neosartora fischeri | L-Alanine | (S)-1 | 24 | 91 |
| E. coli-ArRTA | Arthrobacter sp. | L-Alanine | (S)-1 | 12 | 78 |
| E. coli-AtTA | Aspergillus terreus | Isopropylamine | (S)-1 | 8 | 54 |

As shown in Table 2 and FIG. 2b, several transaminases were able to convert 5a to (R)-1a, and the best result was obtained from E. coli-BmTA expressing the transaminase from Bacillus megaterium. BmTA was able to convert 10 mM of 5a to (R)-1a with 82% efficiency and >99% ee with L-alanine as the amino donor. BmTA was thus chosen for the artificial cascade for the production of (R)-1a.

Similarly, (R)-enantioselective transaminases were searched in database and three transaminase candidates such as NfTA, ArRTA and AtTA, were identified for the examination on the conversion of 5a to (S)-1a. The selected transaminases were cloned in individual pRSFDuet-1 plasmid, and transformed into three different E. coli T7 strains. 10 g cdw/L of resting cells of E. coli strains expressing three (R)-enantioselective transaminases were used for the biotransformation of 10 mM of 5a in KP buffer containing glucose (0.5%) and 200 mM of amino donor (L-alanine or isopropylamine) for 24 h. As shown in Table 2 and in FIG. 2b, only E. coli-NfTA expressing transaminase from Neosartora fischeri was able to convert 5a to (S)-1a with relatively high efficiency. NfTA was able to convert 10 mM of 5a to (S)-1a in 24% and 91% ee with L-alanine as the amino donor. Therefore, NfTA was chosen for the artificial cascade for the production of (S)-1a.

### Example 3. Engineering of E. coli Co-Expressing 5 Enzymes of the Artificial Cascade for the Conversion of Styrene 2a to (R)- and (S)-2-Phenylglycinol 1a

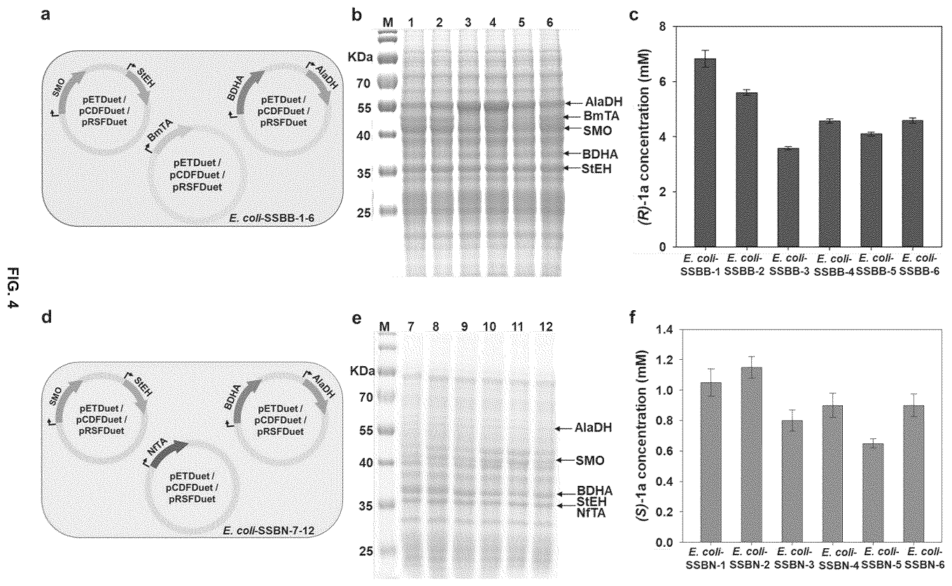

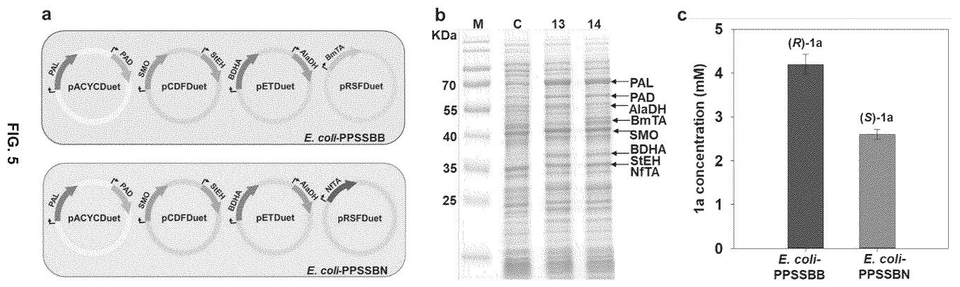

For the efficient whole cell biotransformation of 2a to (R)- and (S)-1a with a single strain, a combinatorial approach was attempted for the expression of enzymes in the artificial cascade. Combinatorial approach facilitates to obtain maximum conversion efficiency by optimizing the expression level of multiple enzymes in the cascade. This could be achieved by expressing the enzymes of the artificial cascade from three plasmids (pCDFDuet-1, pETDuet-1 and pRSFDuet-1) with varying copy numbers of 10 to >100 (N. H. Tolia & L. Joshua-Tor, Nat. Methods 2006, 3, 55-64). Different combinations of these plasmids could result in different expression levels of the enzymes (Y. Zhou, S. Wu & Z. Li, Angew. Chem. 2016, 128, 11819-11822; and S. Wu et al., Adv. Synth. Catal. 2017, 359, 2132-2141). Thus, SMO and StEH for the conversion of 2a to 4a were cloned together in pCDFDuet-1, pETDuet-1 and pRSFDuet-1. BDHA for the conversion of 4a to 5a and alanine dehydrogenase (S. Wu, J. Liu & Z. Li, ACS Catal. 2017, 7, 5225-5233) for recycling amino donor (L-alanine) were cloned together in pCDFDuet-1, pETDuet-1 and pRSFDuet-1. BmTA for (R)-1a or NfTA for (S)-1a production from 5a was cloned in pCDFDuet-1, pETDuet-1 and pRSFDuet-1. The recombinant pCDFDuet-1, pETDuet-1 and pRSFDuet-1 plasmids (FIG. 3) were transformed into twelve different E. coli strains so that all five enzymes were present in compatible plasmids in each E. coli strain (FIG. 4a,d and Table 1). The E. coli strains containing the five-enzyme cascade for the conversion of 2a to (R)-1a and (S)-1a, were named E. coli-SSBB-1-6 and E. coli-SSBN-1-6, respectively. SDS-PAGE analysis of E. coli-SSBB-1-6 and E. coli-SSBN-1-6 were performed, which confirmed the expression of all five enzymes in the artificial cascade (FIGS. 4b,e). The expression of BmTA was relatively higher in E. coli-SSBB-1 and E. coli-SSBB-2 compared to other strains

### Example 4. One-Pot Biotransformation of Styrene 2a to (R)- and (S)-2-Phenylglycinol 1a with E. coli Co-Expressing 5 Enzymes of Artificial Cascade

Cascade conversion of styrene 2a to (R)- and (S)-2- phenylglycinol 1a

Biotransformation was performed at 30° C. in a two-phase system with 2 mL suspension of E. coli-SSBB-1-6 (20 g cdw/L) and E. coli-SSBN-1-6 (20 g cdw/L) in phosphate buffer (200 mM, pH 8) with n-hexadecane containing 2a (final conc., 10 mM for (R)-1a and 5 mM for (S)-1a). Glucose (2%) and 100 mM of L-alanine were added to the reaction mixture at 0 h. Additional glucose (1%) and L-alanine (100 mM) were added together at 12 h and 20 h. The reaction was continued for 24 h and 48 h for the synthesis of (R)- and (S)-1a, respectively. (R)- and (S)-1a was present only in the aqueous phase and 50 μL of sample was taken to analyse its concentration using reverse phase HPLC.

### Results and Discussion

One-pot biotransformation of 10 mM of 2a to (R)-1a was performed with resting cells of E. coli-SSBB-1-6 (20 g cdw/L) at 30° C. for 24 h. As shown in FIG. 4c, the difference in copy numbers of expression vectors showed huge variation in the synthesis of (R)-1a by E. coli-SSBB-1-6. E. coli-SSBB-1 (pCDF-SMO-StEH, pET-BDHA-AlaDH, pRSF-BmTA) was the best strain to produce 6.83 mM of (R)-1a (>99% ee and 68% conversion) from 2a (FIG. 4c). One-pot biotransformation of 5 mM of 2a to (S)-1a was performed with resting cells of E. coli-SSBN-1-6 (20 g cdw/L) at 30° C. for 48 h. The engineered E. coli strains expressing the developed enzyme cascade converted styrene 2a to (S)-1a in 91% ee at 315 mg/L. As shown in FIG. 4f, E. coli-SSBN-2 (pCDF-SMO-StEH, pET-NFTA, pRSF-BDHA-AlaDH) showed the highest conversion of 23%. As NfTA was not efficient like BmTA, a relatively low production of (S)-1a was observed in E. coli-SSBN-1-6. Evolution of NfTA and search for new R-selective transaminase for further improvement of (S)-1a production is under progress.

### Example 5. Optimization of Reaction Conditions for the Conversion of Styrene 2a to Enantiopure 2-Phenylglycinol 1a Using E.coli-SSBB-1

To further improve the conversion of 2a to (R)-1a by E. coli-SSBB-1, the reaction conditions for one-pot biotransformation described in Example 4, were optimized (Table 3).

TABLE 3
Optimization of reaction conditions for the production of (R)-
2-phenylglycinol 1a from styrene 2a using E. coli-SSBB-1.

| | Cell conc. | Organic | Organic phase:KP | | Conv. |
|---|---|---|---|---|---|
| Strain | (g cdw L⁻¹) | phase | buffer ratio | Amino donor | (%) |
| E. coli-SSBB-1 | 20 | n-Hexadecane | 1:1 | L-alanine and | 68% |
| | | | | 100 mM NH₃/NH₄Cl | |
| E. coli-SSBB-1 | 20 | n-Hexadecane | 1:1 | 100 mM NH₃/NH₄Cl | 69% |
| E. coli-SSBB-1 | 20 | Ethyl oleate | 1:1 | 100 mM NH₃/NH₄Cl | 68% |
| E. coli-SSBB-1 | 20 | n-Hexadecane | 0.2:1 | L-alanine and | 72% |
| | | | | 100 mM NH₃/NH₄Cl | |
| E. coli-SSBB-1 | 20 | n-Hexadecane | 0.2:1 | 100 mM NH₃/NH₄Cl | 74% |
| E. coli-SSBB-1 | 20 | Ethyl oleate | 0.2:1 | 100 mM NH₃/NH₄Cl | 72% |

Conversion of Styrene 2a to (R)- 2- Phenylglycinol 1a Using E. coli-SSBB-1

Whole cell biotransformation was performed at 30° C. for 24 h in a two-phase system with 2 mL suspension of E. coli-SSBB-1 (20 g cdw/L) in phosphate buffer (200 mM, pH 8) with different volumes of n-hexadecane or ethyl oleate containing 2a (10 mM, final conc.). Glucose (2%) and 100 mM of an amino donor selected from L-alanine and 100mM NH₃/NH₄Cl, and 100 mM NH₃/NH₄Cl, were added to the reaction mixture at 0 h. Additional glucose (1%) and amino donor (100 mM) were added together at 12 h and 20 h.

### Results and Discussion

Firstly, the biotransformation of 2a to (R)-1a was performed with E. coli-SSBB-1 (20 g cdw/L) with 0 mM or 100 mM supplementation of L-alanine. E. coli naturally produces L-alanine and E. coli-SSBB-1 expresses alanine dehydrogenase for recycling L-alanine using NH₃/NH₄Cl, therefore the supplementation of L-alanine might not be necessary. In fact, same conversion efficiency (68-69%) was obtained with or without L-alanine supplementation. Secondly, the organic phase n-hexadecane was replaced by ethyl oleate, and the synthesis of (R)-1a from 2a was evaluated. The engineered E. coli strains expressing the developed enzyme cascade converted styrene 2a to (R)-1a in >99% ee at 1015 mg/L. As n-hexadecane is obtained from petroleum, a greener and biocompatible organic phase such as ethyl oleate which can be obtained from biodiesel was tested. No significant difference was observed in the conversion (68-69%), suggesting that ethyl oleate could be used as a greener alternative. In addition, as the organic phase could damage the cell membrane of whole cells after long exposure during biotransformation, the volume of organic phase (n-hexadecane or ethyl oleate) was reduced by changing the ratio of organic phase:phosphate buffer ratio from 1:1 to 0.2:1. Surprisingly, improved conversion of 2a to (R)-1a was observed with both n-hexadecane:phosphate buffer and ethyl oleate:phosphate buffer in 0.2:1 ratio, respectively. 74% conversion of 2a to (R)-1a was achieved in biotransformation with n-hexadecane:phosphate buffer in 0.2:1 ratio. Reduced volume of organic phase usage increased the conversion efficiency and could also contribute to reducing operation cost in a large-scale production.

### Example 6. One-Pot Biotransformation of Styrenes 2a-2k and Indene 2l to (R)-Phenylglycinols 1a-1k and (1R,2R)-1-Amino-2-Indanol 1l with E. coli Co-Expressing 5 Enzymes of Artificial Cascade

### Cascade Conversion of Styrenes 2b-l to (R)-2-Phenylglycinols 1b-l

Biotransformation was performed at 30° C. in a two-phase system with 2 mL suspension of E. coli-SSBB-1 in phosphate buffer (200 mM, pH 8) with 0.4 mL of n-hexadecane containing 2b-l (final conc., 5-10 mM). Glucose (2%) and 100 mM of NH₃/NH₄Cl were added to the reaction mixture at 0 h. Additional glucose (1%) and NH₃/NH₄Cl (100 mM) were added together at 12 h and 20 h. After 48 h of biotransformation, (R)-1b-l were present only in the aqueous phase and their concentration were analysed using reverse phase HPLC.

### Results and Discussion

Substituted (R)-phenylglycinols 1a-1k and (1R,2R)-1-amino-2-indanol 1l are also important building blocks in the synthesis of pharmaceutics. Therefore, E. coli-SSBB-1 cells were examined for the conversion of substituted styrenes 2a-k and indene 2l to (R)-1a-1k and (1R,2R)-1-amino-2-indanol 1l. One-pot biotransformation was performed in the two-phase system with n-hexadecane and phosphate buffer (0.2:1) with 5-10 mM of 2a-2l and E. coli-SSBB-1 (20 g cdw/L). The conversion to 1a-1l are listed in Table 4. All the tested substrates (2a-2k) were converted to their respective (R)-phenylglycinols (1a 1k) and (1R,2R)-1-amino-2-indanol 1l in 95->99% ee. Among the eleven substituted substrates, eight substrates were converted to their respective (R)-phenylglycinols with >50% conversion. Specifically, 2c, 2d, 2i, 2k showed >60% conversion. Thus, the developed artificial cascade is useful for the production of various (R)-2-phenylglycinols 1a-1k and (1R,2R)-1-amino-2-indanol 1l.

TABLE 4
Cascade biotransformation of styrenes 2a-2k and indene
2l to (R)-2-phenylglycinols-1a-1k and (1R,2R)-1-amino-2-
indanol 1l with resting cells of E. coli-SSBB-1.

| | | Substrate | | | | |
|---|---|---|---|---|---|---|
| | | conc.^{b)} | Cell conc. | | Conv.^{c)} | ee^{c)} |
| Substrate^{a)} | R | (mM) | (g cdw L⁻¹) | Product | (%) | (%) |
| 2a | H | 10 | 20 | 1a | 74 | >99 |
| 2b | o-F | 10 | 20 | 1b | 57 | >99 |
| 2c | m-F | 10 | 20 | 1c | 69 | >99 |
| 2d | p-F | 10 | 20 | 1d | 73 | 99 |
| 2e | m-Cl | 10 | 20 | 1e | 56 | >99 |
| 2f | p-Cl | 5 | 25 | 1f | 53 | >99 |
| 2g | m-Br | 5 | 25 | 1g | 34 | 95 |
| 2h | p-Br | 5 | 25 | 1h | 22 | >99 |
| 2i | m-Me | 10 | 20 | 1i | 67 | >99 |
| 2j | p-Me | 5 | 25 | 1j | 57 | >99 |
| 2k | p-OMe | 5 | 25 | 1k | 61 | >99 |
| 2l | — | 5 | 25 | 1l | 24 | >99 |

^{a)}Substrates 2a-2l were converted to 1a-1l by E. coli SSBB-1 (20 g cdw/L) at 30° C. for 48 h in a two-phase system of 2 mL phosphate buffer (200 mM, pH 8) and 0.4 mL n-hexadecane containing 2a-2l.
^{b)}normalized to aqueous phase volume.
^{c)}Concentration and enantiomeric excess of 1a-1l were analysed using reverse phase HPLC with SB-C18 and Crownpak CR (+) chiral column, respectively.
Biotransformations were performed in duplicates and the mean values were presented.

### Example 7. Scaled-Up One-Pot Biotransformation of Styrenes 2a, 2d, 2e, 2j, 2k to (R)-2-Phenylglycinols 1a, 1d, 1e, 1j, 1k

One-pot biotransformation of styrenes 2a, 2d, 2e, 2j, 2k to (R)-2-phenylglycinols 1a, 1d, 1e, 1j, 1k as described in Example 6, were scaled up.

Preparative biotransformation of styrenes (2a, 2d, 2e, 2j, 2k) to produce (R)-2-phenylglycinols (1a, 1d, 1e, 1j, 1k) Preparative biotransformation was performed at 30° C. in a two-phase system with 100 mL suspension of E. coli-SSBB-1 (25 g cdw/L) in phosphate buffer (200 mM, pH 8) and 20 mL of n-hexadecane containing 50 mM of 2a, 2d, 2e, 2j, 2k. Glucose (2%) and NH₃/NH₄Cl (100 mM) were added to the reaction at 0 h, 16 h and 24 h. After 48 h of biotransformation, the aqueous phase was separated by centrifugation at 4200× g for 10 min. The aqueous phase was saturated with NaCl, adjusted to pH<2 with HCl (10 M), and washed with ethyl acetate (2×30 mL) to remove trace n-hexadecane and other organic impurities. The aqueous phase was adjusted to pH>12 with NaOH (10 M), followed by extraction with ethyl acetate (3×100 mL). The organic phase was separated and dried over Na₂SO₄. After filtration, the organic phase was subjected to evaporation. The crude phenylglycinols were purified by flash chromatography on a silica gel column with CH₂Cl₂:MeOH:NH₃ (28% aqueous solution) of 100:10:1 as eluent (R_{f}=0.3-0.6 for (R)-1a, 1d, 1e, 1j and 1k). The collected fraction containing the product was dried over Na₂SO₄. After filtration, the organic solvent was removed by evaporation, and the product was dried under vacuum overnight to obtain the pure product.

### Results and Discussion

Preparative biotransformation was performed with resting cells of E. coli-SSBB-1 (25 g cdw/L) in 100 mL of phosphate buffer with 20 mL of n-hexadecane containing 2a, 2d, 2e, 2j, 2k (50 mM, normalized to aqueous phase volume). After reaction for 48 h at 30° C. , (R)-1a, 1d, 1e, 1j and 1k were isolated from the aqueous phase in high purity with 59%, 50%, 37%, 32%, and 44% yield, respectively.

### 1a

81 mg, 59% yield, HPLC purity: >98%. ¹H NMR (400 MHZ, CDCl₃) δ 7.44-7.21 (m, 5H), 4.04 (dd, J=8.4, 4.3 Hz, 1H), 3.73 (dd, J=10.9, 4.3 Hz, 1H), 3.56 (dd, J=10.9, 8.4 Hz, 1H). ¹³C NMR (101 MHZ, CDCl₃) δ 142.57, 128.84, 127.75, 126.70, 68.02, 57.55.

### 1d

78 mg, 50% yield, HPLC purity: >98%. ¹H NMR (400 MHZ, CDCl₃) δ 7.36-7.23 (m, 2H), 7.08-6.96 (m, 2H), 4.05 (dd, J=8.2, 4.3 Hz, 1H), 3.70 (dd, J=10.8, 4.3 Hz, 1H), 3.53 (dd, J=10.8, 8.3 Hz, 1H). ¹³C NMR (101 MHZ, CDCl₃) 0 162.36 (d, J=245.8 Hz), 128.33 (d, J=8.0 Hz), 139.69, 115.66 (d, J=21.3 Hz), 68.00, 56.85.

### 1e

64 mg, 37% yield, HPLC purity: >98%. ¹H NMR (400 MHZ, CDCl₃) δ 7.40-7.14 (m, 4H), 4.03 (dd, J=7.2, 4.0 Hz, 1H), 3.73 (dd, J=10.6, 3.6 Hz, 1H), 3.53 (dd, J=10.4, 8.1 Hz, 1H). ¹³C NMR (101 MHZ, CDCl₃) δ 145.03, 134.72, 130.10, 127.86, 126.96, 124.94, 68.03, 57.14.

### 1j

49 mg, 32% yield, HPLC purity: >98%. ¹H NMR (400 MHZ, CDCl₃) δ 7.18 (dd, J=23.7, 8.1 Hz, 4H), 4.01 (dd, J=8.4, 4.3 Hz, 1H), 3.71 (dd, J=10.9, 4.3 Hz, 1H), 3.54 (dd, J=10.8, 8.5 Hz, 1H), 2.33 (s, 3H). ¹³C NMR (101 MHZ, CDCl₃) δ 139.53, 137.43, 129.51, 126.59, 68.02, 57.25, 21.24.

### 1k

73 mg, 44% yield, HPLC purity: >98%. ¹H NMR (400 MHZ, CDCl₃) δ 7.30-7.20 (m, 2H), 6.92-6.84 (m, 2H), 4.00 (dd, J=8.0, 4.1 Hz, 1H), 3.79 (s, 3H), 3.69 (dd, J=10.8, 4.3 Hz, 1H), 3.59-3.48 (m, 1H). ¹³C NMR (101 MHZ, CDCl₃) δ 159.03, 127.63, 114.05, 100.00, 67.86, 56.72, 55.30.

### Example 8. Design of Cascade Biotransformations and Development of Resting-Cells Bioprocess to Convert Bio-Based L-Phenylalanine 6 to (R)- and (S)-2-Phenylglycinol 1a

For the conversion of bio-based L-phenylalanine 6 to enantiopure (R)- and (S)-1a (FIG. 1b), artificial enzyme cascade consisting of decarboxylation-deamination-epoxidation-hydrolysis-oxidation-transamination, PAL of Arabidopsis thaliana and PAD of Saccharomyces cerevisiae were expressed together in the cascade for 2a to (R)- and (S)-1a.

### Cascade Conversion of L-Phenylalanine 6 to (R)- and (S)-Phenylglycinol 1a

Biotransformation of L-phenylalanine 6 using E. coli-PPSSBB and E. coli-PPSSBN was performed at 30° C. for 24 h for the synthesis of (R)- and (S)-1a, respectively. Reaction was performed in a two-phase system with 5 mL suspension of E. coli-PPSSBB or E. coli-PPSSBN (20 g cdw/L) in phosphate buffer (200 mM, pH 8) with 1 mL of n-hexadecane. 6 (10 mM), glucose (2%) and NH₃/NH₄Cl (100 mM) were added to the reaction at 0 h. Additional glucose (1%) and NH₃/NH₄Cl (100 mM) were added together at 12 h. At 24 h, 50 μL of the aqueous phase was collected to quantify the concentration of (R)- or (S)-1a using reverse phase HPLC.

### Results and Discussion

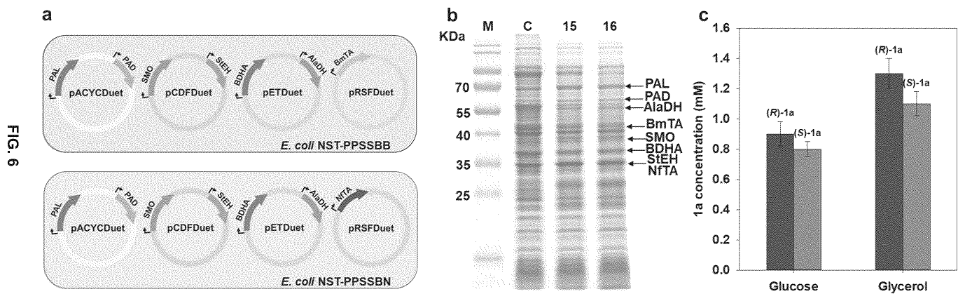

We previously proved that PAL and PAD efficiently produces 2a from 6 (139 mM of 2a, 92% conversion, B. R. Lukito et al., Adv. Synth. Catal. 2019, 361, 3560-3568; B. R. Lukito et al., ChemCatChem 2019, 11, 831-840; Y. Zhou, S. Wu & Z. Li, Angew. Chem. 2016, 128, 11819-11822; and Y. Zhou et al., ChemSusChem 2018, 11, 2221-2228). Thus, PAL and PAD enzymes cloned in pACYCDuet-1 (pACYC-PAL-PAD, B. R. Lukito et al., ChemCatChem 2019, 11, 831-840) were co-expressed in E. coli-SSBB-1 (pCDF-SMO-StEH, pET-BDHA-AlaDH, pRSF-BmTA) and E. coli-SSBN-7 (pCDF-SMO-StEH, pET-BDHA-AlaDH, pRSF-NfTA), and the resulted recombinant strains were named as E. coli-PPSSBB and E. coli-PPSSBN, respectively (FIG. 5a). SDS-PAGE analysis of the crude cell lysate of E. coli-PPSSBB and E. coli-PPSSBN was performed (FIG. 5b), showing the expression of all seven enzymes of the artificial cascade in E. coli-PPSSBB and E. coli-PPSSBN.

Cascade biotransformation of 10 mM of 6 to (R)- and (S)-1a was performed with the resting cells (20 g cdw/L) of E. coli-PPSSBB and E. coli-PPSSBN, respectively, in the two-phase system with n-hexadecane and phosphate buffer (0.2:1). After 24 h of reaction at 30° C. , 576 mg/L of (R)-1a (42% conversion efficiency) and 356 mg/L of (S)-1a (26% conversion efficiency) was produced by E. coli-PPSSBB and E. coli-PPSSBN, respectively (FIG. 5c). This is the first report on the synthesis of (R)- and (S)-1a from bio-based substrate 6. Since the accumulation of intermediates such as 4a and 5a was observed, further enhancement of the productivity might be achieved by improving the activity of the alcohol dehydrogenase and transaminase.

### Example 9. Design of Cascade Biotransformations and Development of Growing-Cells Bioprocess to Convert Renewable Feedstocks Glucose 8 and Glycerol 9 to (R)- and (S)-2-Phenylglycinol 1a

E. coli naturally produces L-phenylalanine 6 by fermentation of low-cost renewable feedstocks such as glucose 8 and glycerol 9 through the Shikimate pathway (B. S. Sekar, B. R. Lukito & Z. Li, ACS Sustain. Chem. Eng. 2019, 7, 12231-12239; M. Weiner et al., Biochem. Eng. J. 2014, 83, 62-69; and Y. Liu et al., BMC Biotechnol. 2018, 18, 5). The natural L-phenylalanine biosynthesis pathway was combined with the artificial enzyme cascade of converting 6 to (R)-or (S)-1a (developed in Example 8), to transform 8 and 9 to (R)- and (S)-1a, respectively (FIG. 1c). As the biosynthesis of 6 is strictly regulated in E. coli by feedback regulation (S .- P. Liu et al., Process Biochem. 2013, 48, 413-419), we engineered E. coli NST74 (DE3) containing natural L-phenylalanine biosynthesis pathway for the overproduction of 6 from 8 or 9 with relaxed feedback regulation (B. S. Sekar, B. R. Lukito & Z. Li, ACS Sustain. Chem. Eng. 2019, 7, 12231-12239; and Y. Zhou et al., ChemSusChem 2018, 11, 2221-2228). This strain was used as the host.

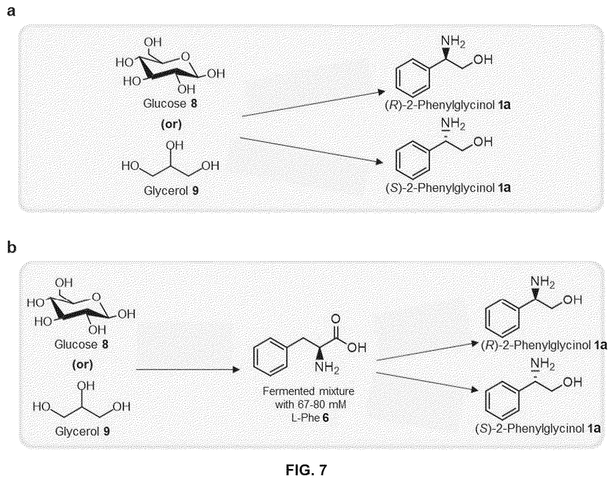

The plasmids pACYC-PAL-PAD, pCDF-SMO-StEH, pET-BDHA-AlaDH, pRSF-BmTA and pACYC-PAL-PAD, pCDF-SMO-StEH, pET-BDHA-AlaDH, pRSF-NfTA were transformed into E. coli NST74 (DE3) to produce E. coli NST-PPSSBB and E. coli NST-PPSSBN, respectively (FIG. 6a). SDS-PAGE analysis was performed with the crude cell lysate of E. coli NST-PPSSBB and E. coli NST-PPSSBN (FIG. 6b). The expression of all seven enzymes in the artificial cascade were confirmed in E. coli NST-PPSSBB and E. coli NST-PPSSBN.

### Fermentation of Glucose 8 and Glycerol 9 to Produce (R)- or (S)-2-Phenylglycinol 1a

E. coli NST-PPSSBB and E. coli NST-PPSSBN were inoculated, respectively, in 2 mL of LB medium containing appropriate antibiotics (50 μg/mL kanamycin, 25 μg/mL chloramphenicol, 50 μg/mL streptomycin, and 100 μg/mL ampicillin) and grown at 37° C., 220 rpm for 6-8 h. 1 mL of the culture of E. coli NST-PPSSBB or E. coli NST-PPSSBN was added to modified M9 medium (50 mL) with 20 g/L of 8 or 9, respectively, and appropriate antibiotics (50 μg/mL kanamycin, 25 μg/mL chloramphenicol, 50 μg/mL streptomycin, and 100 μg/mL ampicillin) in a 250 mL flask. After 2 h of growth at 37° C. , IPTG (0.1 mM) and n-hexadecane (10 mL) were added and the growth temperature was set at 25° C. At 24 h, 50 μL of the aqueous phase was collected to quantify the concentration of (R)- or (S)-1a using reverse phase HPLC.

### Results and Discussion

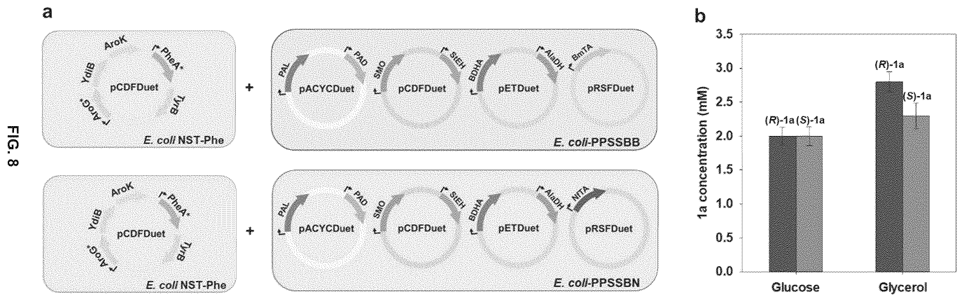

The fermentation of 8 or 9 to (R)- and (S)-1a was performed with E. coli NST-PPSSBB and E. coli NST-PPSSBN, respectively, in 50 mL of modified M9 media containing 10 g/L of 8 or 9, and 10 mL of n-hexadecane at 25° C. for 24 h (FIG. 7a). E. coli NST-PPSSBB produced 123 and 178 mg/L of (R)-1a from glucose 8 and glycerol 9, respectively, while E. coli NST-PPSSBN produced 110 and 151 mg/L of (S)-1a from 8 and 9, respectively (FIG. 6c). This is the first report on the bioproduction of (R)- and (S)-1a from low-cost renewable feedstocks such as 8 and 9. However, further optimization of the reaction conditions and engineering of strains is necessary to improve the fermentative production and explore the possibilities of large-scale production, and is addressed in the following example.

### Example 10. Production of (R)- and (S)-2-Phenylglycinol 1a from Renewable Feedstocks Glucose 8 and Glycerol 9 by Coupling Two Strains Expressing L-Phenylalanine Biosynthesis Pathway and the Artificial Cascade

The fermentative synthesis of (R)- and (S)-1a from sugars 8 and 9 could burden the cells due to overexpression of several enzymes in a single strain. In addition, the supplementation of organic phase to growing cells and the accumulation of reaction intermediates could inhibit the cell growth, thus reducing the fermentative synthesis of (R)- and (S)-1a. To overcome these issues, we coupled fermentation-biotransformation (B. R. Lukito et al., Adv. Synth. Catal. 2019, 361, 3560-3568; B. S. Sekar, B. R. Lukito & Z. Li, ACS Sustain. Chem. Eng. 2019, 7, 12231-12239; and Y. Zhou et al., Biotechnol. Bioeng. 2020, 117, 2340-2350) to fermentatively produce high concentrations of 6 from 8 or 9 and sequentially biotransform 6 to (R)- and (S)-1a, respectively.

Coupled Fermentation-Biotransformation for the Production of (R)- or (S)-2-Phenylglycinol 1a from Glucose 8 or Glycerol 9

Fermentative production of L-phenylalanine 6 from 8 or 9 by E. coli NST-Phe was performed as mentioned previously (B. S. Sekar, B. R. Lukito & Z. Li, ACS Sustain. Chem. Eng. 2019, 7, 12231-12239), to achieve 67-80 mM of 6, and the fermentation culture containing biosynthesized 6 and E.coli NST-Phe cells was stored at 4° C. or used directly in biotransformation. 5 mL of reaction mixture was prepared with 5 mL of fermentation broth containing biosynthesized 6 (final conc. 10 mM), phosphate buffer (200 mM, pH 8), glucose (2%), and NH₃/NH₄Cl (100 mM). Resting cells of E. coli NST-PPSSBB or E. coli NST-PPSSBN (20 g cdw/L) and 1 mL of n-hexadecane were added, and biotransformation was performed at 30° C. and 220 rpm. Additional glucose (1%) and NH₃/NH₄Cl (100 mM) were added together at 12 h. At 24 h, 50 μL of the aqueous phase was collected to quantify the concentration of (R)- or (S)-1a using reverse phase HPLC. Experiment was performed in duplicates with error bars showing ±SD.

### Results and Discussion

Fermentative production of L-phenylalanine 6 from sugars 8 and 9 was performed by E. coli NST-Phe to obtain high concentrations of 6, followed by the one-pot biotransformation of 6 to
(R)- and (S)-1a by the resting cells of E. coli-PPSSBB and E. coli-PPSSBN, respectively (FIGS. 7b and 8a).

E. coli NST-Phe overexpressing five key enzymes of Shikimate pathway (AroG*, Arok, YdiB, PheA*, and TyrB) was employed to produce 67-80 mM of 6 (B. R. Lukito et al., Adv. Synth. Catal. 2019, 361, 3560-3568; and B. S. Sekar, B. R. Lukito & Z. Li, ACS Sustain. Chem. Eng. 2019, 7, 12231-12239). Resting cells (20 g cdw/L) of E. coli-PPSSBB and E. coli-PPSSBN were added to the fermentation broth with biosynthesized 6, to perform biotransformation of 6 to (R)- and (S)-1a, respectively. By coupled fermentation-biotransformation approach, 274 and 384 mg/L of (R)-1a were obtained from glucose 8 and glycerol 9, respectively. For the synthesis of (S)-1a, the product titer of 274 and 301 mg/L were achieved from glucose 8 and glycerol 9, respectively (FIG. 8b). Table 5 summarizes the production of (R)- and (S)-1a from glucose 8 and glycerol 9.

TABLE 5
Bioproduction of (R)- and (S)-2-Phenylglycinol 1a from renewable resources
such as L-phenylalanine (L-Phe) 6, glucose 8, and glycerol 9.

| | Renewable | | | Conc. |
|---|---|---|---|---|
| Experiment | Substrate | Strain | Product | (mM) |
| Resting cells biotransformation ^{a} | L-Phe 6 | E. coli-PPSSBB | (R)-1 | 4.2 |
| Resting cells biotransformation ^{a} | L-Phe 6 | E. coli-PPSSBN | (S)-1 | 2.6 |
| Growing cells fermentation ^{b} | Glucose 8 | E. coli NST-PPSSBB | (R)-1 | 1 |
| Growing cells fermentation ^{b} | Glycerol 9 | E. coli NST-PPSSBB | (R)-1 | 1.3 |
| Growing cells fermentation ^{b} | Glucose 8 | E. coli NST-PPSSBN | (S)-1 | 1 |
| Growing cells fermentation ^{b} | Glycerol 9 | E. coli NST-PPSSBN | (S)-1 | 1.1 |
| Coupled fermentation-biotransformation | Glucose 8 | E. coli NST-Phe + | | |
| ^{c} | | E. coli-PPSSBB | (R)-1 | 2 |
| Coupled fermentation-biotransformation | Glycerol 9 | E. coli NST-Phe + | (R)-1 | 2.8 |
| ^{c} | | E. coli-PPSSBB | | |
| Coupled fermentation-biotransformation | Glucose 8 | E. coli NST-Phe + | | |
| ^{c} | | E. coli-PPSSBN | (S)-1 | 2 |
| Coupled fermentation-biotransformation | Glycerol 9 | E. coli NST-Phe + | (S)-1 | 2.2 |
| ^{c} | | E. coli-PPSSBN | | |

^{a} see Example 8 for experimental protocol.
^{b} see Example 9 for experimental protocol.
^{c} see Example 10 for experimental protocol.

The developed strategies are potentially useful for producing high-value chemicals from cheap and renewable feedstocks.

1. A method for producing an enantiomerically pure or enantiomerically enriched (R)- or (S)-2-phenylglycinol or a derivative thereof using one or more recombinant microbial cells genetically engineered to overexpress multiple enzymes, which method comprises subjecting styrene or a derivative thereof to multiple enzyme-catalyzed chemical transformations in a one-pot reaction system.

2. The method of claim 1, wherein the method produces enantiomerically pure or enantiomerically enriched 2-phenylglycinol or a derivative thereof, comprising the steps of:
(a) generating phenylethane diol or a derivative thereof from styrene or a derivative thereof by conducting an epoxidation reaction catalyzed by an epoxidase to form styrene oxide or a derivative thereof and conducting a hydrolysis reaction catalyzed by an epoxide hydrolase on the epoxide, or by a dihydroxylation reaction catalyzed by a dioxygenase:
(b) generating 2-hydroxyacetophenone or a derivative thereof from the phenylethane diol or a derivative thereof by an oxidation reaction catalyzed by an alcohol dehydrogenase or a secondary alcohol oxidase; and
(c) generating enantiomerically pure or enantiomerically enriched 2-phenylglycinol or a derivative thereof from 2-hydroxyacetophenone or a derivative thereof by a transamination reaction catalyzed by a transaminase or a reductive amination reaction catalyzed by an amine dehydrogenase.

3. The method according to claim 2, wherein one or more of the following apply:
(a) the epoxidase is a styrene monooxygenase;
(b) the epoxide hydrolase, when used, is from Solanum tuberosum or its mutants or similar enzymes with more than 50% identity:
(c) the alcohol dehydrogenase, when used, is from Candida parapsilosis or, more preferably, Bacillus subtilis or their mutants or similar enzymes with more than 50% identity: and/or
(d) the transaminase is from Bacillus megaterium, Chromobacterium violaceum, Vibrio fluvialis, Martelella mediterranea, Neosartora fischeri, Arthrobacter sp., or Aspergillus terreus or their mutants or similar enzymes with more than 50% identity.

4. (canceled)

5. The method according to claim 1, wherein one of the one or more recombinant microbial cells genetically engineered to overexpress multiple enzymes (i.e. the one or more recombinant microbial cells genetically engineered to overexpress multiple enzymes) is selected from one or more of the group consisting of:
(a) E. coli T7-pCDF-SMO-StEH, pET-BDHA-AlaDH, pRSF-BmTA;
(b) E. coli T7-pCDF-SMO-StEH, pET-BmTA, pRSF-BDHA-AlaDH;
(c) E. coli T7-pCDF-BDHA-AlaDH, pET-SMO-StEH, pRSF-BmTA;
(d) E. coli T7-pCDF-BDHA-AlaDH, pET-BmTA, pRSF-SMO-StEH;
(e) E. coli T7-pCDF-BmTA, pET-SMO-StEH, pRSF-BDHA-AlaDH; and (f) E. coli T7-pCDF-BmTA, pET-BDHA-AlaDH, pRSF-SMO-StEH.

6. (canceled)

7. The method according to claim 1, wherein one of the one or more recombinant microbial cells genetically engineered to overexpress multiple enzymes (i.e. the one or more recombinant microbial cells genetically engineered to overexpress multiple enzymes) is selected from one or more of the group consisting of:
(a) E. coli T7-pCDF-SMO-StEH, pET-BDHA-AlaDH, pRSF-NFTA;
(b) E. coli T7-pCDF-SMO-StEH, pET-NFTA, pRSF-BDHA-AlaDH;
(c) E. coli T7-pCDF-BDHA-AlaDH, pET-SMO-StEH, pRSF-NFTA;
(d) E. coli T7-pCDF-BDHA-AlaDH, pET-NFTA, pRSF-SMO-StEH;
(e) E. coli T7-pCDF-NFTA, pET-SMO-StEH, pRSF-BDHA-AlaDH; and
(f) E. coli T7-pCDF-NFTA, pET-BDHA-AlaDH, pRSF-SMO-StEH.

8. The method according to claim 1, wherein one of the one or more recombinant microbial cells genetically engineered to overexpress multiple enzymes (i.e. the one or more recombinant microbial cells genetically engineered to overexpress multiple enzymes) is E. coli T7-pCDF-SMO-StEH, pET-NFTA, pRSF-BDHA-AlaDH.

9. The method according to claim 1, wherein the method further comprises providing styrene or a derivative thereof by generating trans-cinnamic acid or a derivative thereof from L-phenylalanine or a derivative thereof by a deamination reaction catalyzed by an ammonia lyase and generating styrene or a derivative thereof from the trans-cinnamic acid or a derivative thereof in a decarboxylation reaction catalyzed by a decarboxylase.

10. (canceled)

11. The method according to claim 1, wherein one of the one or more recombinant microbial cells genetically engineered to overexpress multiple enzymes (i.e. the one or more recombinant microbial cells genetically engineered to overexpress multiple enzymes) is E. coli T7-pACYC-PAL-PAD, pCDF-SMO-SIEH, pET-BDHA-AlaDH, pRSF-BmTA.

12. The method according to claim 1, wherein one of the one or more recombinant microbial cells genetically engineered to overexpress multiple enzymes (i.e. the one or more recombinant microbial cells genetically engineered to overexpress multiple enzymes) is E. coli T7-pACYC-PAL-PAD, pCDF-SMO-StEH, pET-BDHA-AlaDH, pRSF-NfTA.

13. The method according to claim 9, wherein the method further comprises cells that overexpress the natural L-phenylalanine biosynthetic pathway, which cells convert glucose or glycerol to L-phenylalanine.

14-15. (canceled)

16. The method according to claim 13, wherein the microbial cells producing L-phenylalanine from glucose or glycerol that overexpress at least one enzyme is E. coli NST74-pACYC-PAL-PAD, pCDF-SMO-StEH, pET-BDHA-AlaDH, pRSF-BmTA or E. coli NST74-pACYC-PAL-PAD, pCDF-SMO-StEH, pET-BDHA-AlaDH, pRSF-NfTA.

17. The method according to claim 13, wherein the microbial cells producing L-phenylalanine from glucose or glycerol that overexpress at least one enzyme is a combination of E. coli NST74-Phe with E. coli T7-pACYC-PAL-PAD, pCDF-SMO-StEH, pET-BDHA-AlaDH, pRSF-BmTA or is a combination of E. coli NST74-Phe with E. coli T7-pACYC-PAL-PAD, pCDF-SMO-StEH, pET-BDHA-AlaDH, pRSF-NfTA.

18-20. (canceled)

21. An isolated nucleic acid molecule encoding at least one heterologous catalytic enzyme selected from the group comprising:
(a) an epoxidase for conducting an epoxidation reaction to form styrene oxide or a derivative thereof from styrene or a derivative thereof and an epoxide hydrolase for conducting a hydrolysis reaction on the epoxide to provide phenylethanediol or a derivative thereof, or an oxygenase for generating phenylethanediol or a derivative thereof from styrene or a derivative thereof by a dihydroxylation reaction:
(b) an alcohol dehydrogenase or a secondary alcohol oxidase for generating 2-hydroxyacetophenone or a derivative thereof from phenylethanediol by an oxidation reaction:
(c) a transaminase or an amine dehydrogenase for generating enantiomerically pure or enantiomerically enriched 2-phenylglycinol or a derivative thereof by a transamination reaction or a reductive amination from 2-hydroxyacetophenone or a derivative thereof; of (d) an ammonia lyase for generating trans-cinnamic acid or a derivative thereof from L-phenylalanine or a derivative thereof by a deamination reaction; and
(e) a decarboxylase for generating styrene or a derivative thereof from trans-cinnamic acid or a derivative thereof in a decarboxylation reaction.

22-23. (canceled)

24. An expression construct comprising at least one nucleic acid molecule as described in claim 21.

25. One or more recombinant prokaryotic or eukaryotic cells selected from the group comprising bacterial cells, yeast cells, mammalian cells and insect cells, wherein said cells comprise at least one expression construct as described in claim 24.

26. The one or more recombinant prokaryotic or eukaryotic cells according to claim 25, wherein said cells are recombinant bacterial cells.

27. The one or more recombinant prokaryotic or eukaryotic cells according to claim 25, wherein said enzymes have at least 50% amino acid identity with at least one enzyme selected from the group comprising:
(a) a styrene monooxygenase from Pseudomonas sp. VLB120:
(b) an epoxide hydrolase from Solanum tuberosum:
(c) an alcohol dehydrogenase from Candida parapsilosis or, more preferably, Bacillus subtilis (e.g. the epoxide hydrolase is selected from secondary alcohol dehydrogenase from Candida parapsilosis or, more preferably, (R.R)-butanediol dehydrogenase (BDHA));
(d) a transaminase from Bacillus megaterium, Chromobacterium violaceum, Vibrio fluvialis, Martelella mediterranea, Neosartora fischeri, Arthrobacter sp., or Aspergillus terreus (e.g. the transaminase is selected from Bacillus megaterium, Vibrio fluvialis, or Neosartora fischeri):
(e) a phenylalanine ammonia lyase from Arabidopsis thaliana:
(f) a phenylacrylic acid decarboxy lase from Aspergillus niger.

28. The one or more recombinant cells of claim 27, wherein said cells are selected from:
(a) E. coli T7-pCDF-SMO-StEH, pET-BDHA-AlaDH, pRSF-BmTA;
(b) E. coli T7-pCDF-SMO-StEH, pET-BmTA, pRSF-BDHA-AlaDH;
(c) E. coli T7-pCDF-BDHA-AlaDH, pET-SMO-StEH, pRSF-BmTA;
(d) E. coli T7-pCDF-BDHA-AlaDH, pET-BmTA, pRSF-SMO-StEH;
(e) E. coli T7-pCDF-BmTA, pET-SMO-StEH, pRSF-BDHA-AlaDH;
(f) E. coli T7-pCDF-BmTA, pET-BDHA-AlaDH, pRSF-SMO-StEH.
(g) E. coli T7-pCDF-SMO-StEH, pET-BDHA-AlaDH, pRSF-NFTA:
(g) E. coli T7-pCDF-SMO-StEH, pET-NFTA, pRSF-BDHA-AlaDH;
(i) E. coli T7-pCDF-BDHA-AlaDH, pET-SMO-StEH, pRSF-NFTA;
(j) E. coli T7-pCDF-BDHA-AlaDH, pET-NFTA, pRSF-SMO-StEH;
(k) E. coli T7-pCDF-NFTA, pET-SMO-StEH, pRSF-BDHA-AlaDH; and
(1) E. coli T7-pCDF-NFTA, pET-BDHA-AlaDH, pRSF-SMO-StEH.
(m) E. coli T7-pACYC-PAL-PAD, pCDF-SMO-StEH, pET-BDHA-AlaDH, pRSF-BmTA;
(n) E. coli T7-pACYC-PAL-PAD, pCDF-SMO-StEH, pET-BDHA-AlaDH, pRSF-NfTA;
(o) E. coli NST74-pACYC-PAL-PAD, pCDF-SMO-StEH, pET-BDHA-AlaDH, pRSF-BmTA; and
(p) E. coli NST74-pACYC-PAL-PAD, pCDF-SMO-StEH, pET-BDHA-AlaDH, pRSF-NfTA.

29. (canceled)

30. A kit comprising at least one isolated nucleic acid according to claim 21.

31. The method according to claim 1, wherein at least one overexpressed enzyme is located on one or more plasmids or integrated in the chromosome of each of the one or more recombinant microbial cells.


SEQUENCE LISTING (13 sequences)

SEQ ID NO: 1 - DNA, 1041 bp, Artificial Sequence
  BDHA
atgaaggcagcaagatggcataaccaaaaggatatccgtattgaacatatcgaagagccaaaaacggagccgggaaaagtaaagatcaaagtcaaatggtgcggcatctgcggaagtgatttacacgaatatctgggcggcccgatctttattccggttgacaaaccgcacccattaacaaatgaaacggcacctgtcacaatggggcatgaattctccggtgaagttgtcgaagtcggagaaggcgttgaaaattataaagttggagaccgcgttgtagtcgagccgatttttgctacacacggccaccaaggcgcctacaaccttgatgaacaaatgggattcctcggcttagccggcggaggcggcggtttctctgaatacgtctctgtggatgaagagcttttgttcaaacttcctgatgaattatcatatgaacaaggcgcgctcgttgaaccttctgcagttgctctatacgctgtccgctcaagcaaactcaaagcaggcgacaaagcggctgtattcggctgcggcccgatcggacttcttgtcattgaagcgctgaaggctgccggtgcaactgatatttacgctgttgagctttctcctgaacgccagcaaaaagctgaggagcttggcgcgatcatcgttgatccgtctaaaacagacgatgtagtcgctgagattgcagaacgtacaggaggcggtgttgacgtagcattcgaagtcactggtgtcccagtggtgttacgacaagccatccagtccactacaattgccggtgaaaccgtcatcgtcagcatttgggaaaaaggtgctgaaatccatccgaacgatatcgtaatcaaagaacgtacagtaaaaggaattatcggataccgcgacatcttcccggctgtattgtcattaatgaaagaaggctatttctcagccgacaaactcgtaacgaaaaaaatcgtactagatgatttgatcgaggaaggcttcggggctcttattaaagagaaaagccaagtcaaaatccttgttagacctaactaa

SEQ ID NO: 2 - DNA, 1011 bp, Artificial Sequence
  CpSADH
atgtcaatcccgtcgtcccaatacggctttgtcttcaataaacagtctggtctgaatctgcgtaatgatctgccggtccataaaccgaaagccggccagctgctgctgaaagtcgatgcagtgggtctgtgtcattctgacctgcacgtgatttatgaaggcctggattgcggtgacaactacgttatgggccatgaaattgcaggtaccgttgcagcagtcggtgatgacgtcatcaactataaagtgggtgatcgtgtggcgtgtgttggcccgaatggttgcggcggttgtaaatactgccgcggcgctatcgataacgtgtgcaaaaatgcgtttggtgattggttcggcctgggttatgacggcggttatcagcaatacctgctggttacccgtccgcgcaacctgagccgtattccggataatgtctctgctgacgtggcagctgcgagtaccgatgcggtcctgacgccgtatcacgccatcaaaatggcacaggtttcaccgacctcgaacattctgctgatcggtgccggcggtctgggcggtaatgcaattcaagtggccaaagcatttggtgcgaaagttacggtcctggataagaaaaaagaagcgcgtgatcaggcgaaaaaactgggcgctgatgcggtttatgaaaccctgccggaaagcatttctccgggtagtttttccgcctgttttgatttcgtgtcagttcaggcaacgttcgacgtttgccaaaaatacgtcgaaccgaaaggcgttatcatgccggtcggtctgggtgctccgaacctgtcgtttaatctgggtgatctggcgctgcgtgaaattcgcatcctgggcagcttctggggtaccacgaacgacctggatgacgtgctgaaactggtttccgaaggcaaagtgaaaccggtggttcgttcagccaaactgaaagaactgccggaatatatcgaaaaactgcgtaacaatgcatacgaaggtcgcgtcgtctttaacccgtaa

SEQ ID NO: 3 - DNA, 966 bp, Artificial Sequence
  HnTA
atgctgacctttcagaaagttctgaccggttttcagacccgtgcagatgcacgtgcagaacgtaccgatgcatttgcagatggtattgcatggattgaaaatgaatttgtgccgattggcaaagcacgtattccgattctggatcagggttttctgcatagcgatctgacctatgatgttccggcagtttggaatggtcgtatttttcgtctggatgatcatctggatcgtctggaagttagctgtgcaaaaatgcgtctgccgctgccgattgcacgtccggaactgcgtcgtctggttatggaactggttagccgtagcggtctgcgtgatgcctatgttgaaattattgttacccgtggcctgaaatttctgcgtggtgcacaggcagaagatattattccgaatctgtatctgatggccgttccgtatgtttggattctgccgctggaatatcagaatcatggtgcaccggcagttgttacccgtaccgttcgtcgtacaccgccgggtgcactggacccgaccatcaaaaatctgcaatggggtgatctggttcgtggtctgatggaagccggtgatcgtgatagcttttttccgattctgccggatggtgatggtaatgcaaccgaaggtgcaggctataacattgttctggttcgtaatggcgaactgcatacaccgcgtcgtggtgttctggaaggtattacccgtcgtaccgttctggaaattgcagcagcacgtggcctgaaaacacatgttaccgaaattccgattcaggcactgtatgaatgtgatgaactgtttatgtgtagcaccgcaggcggtattatgccgctggttctgctggatggtaatattgttggtgatggcaccgttggtccggttacccgtatgatttgggaagcatattgggatctgcatgatgatccgcagctgagcgaaccggttacctatgcaccgtaa

SEQ ID NO: 4 - DNA, 1362 bp, Artificial Sequence
  DpgTA
atgagtattctgaacgactacaagcgtaaaacggaaggctcggtcttctgggcgcagcgtgcccgcagcgtgatgccggacggtgtgaccgccgacacgcgtgtgtttgatccgcatggcctgttcatcagcgatgcacagggtgttcacaaaaccgatgttgacggcaacgtctatctggatttctttggcggtcatggtgcgctggtgctgggtcatggccacccgcgcgtgaatgcagcaattgcagaagccctgtcacatggcgtgcaatacgcagcttcgcacccgctggaagtgcgttgggccgaacgcatcgttgcggcctttccgagcattcgtaaactgcgcttcaccggctctggtacggaaaccacgctgctggcactgcgtgtggcacgcgctttcaccggtcgtcgcatgattctgcgtatcgccacgcattatcacggttggcatgactttagcgcatctggctacaacagccacttcgatggtcagccggcaccgggtgttctgccggaaatcgcaaagaataccctgctgatccgtccggatgacattgagggtatgcgcgaagtctttgctcaacatggcagtgatattgcagcttttattgcggaaccggtgggctcccacttcggtgtgaccccggttagcgattcctttctgcgtgaaggtgctgaactggcgcgccagtatggcgcactgttcatcctggatgaagttattagcggctttcgtgtcggcaatcatggcatgcaggcgctgctggatgttcaaccggacctgacctgcctggcaaaagcgagcgcgggcggcctgccgggcggtattctgggcggtcgcgaagacgtcatgggcgtgctgtcacgtggttcggatcgcaaggttctgcaccagggcacctttacgggtaacccgatcaccgcggcggcggcgattgcagccatcgacacgattctggaagatgacgtctgtgccaaaattaatgatctgggtcaattcgcccgtgaagcaatgaaccacctgtttgctcgcaagggcctgaattggctggcgtacggccgtttttcaggtttccacctgatgccgggtctgccgccgaacaccacggacaccggctcgatcacgcgtgctgaagtcgcacgtccggatgtgaaaatgattgcagctatgcgcatggcgctgatcctggaaggcgttgatattggcggtcgtggtagcgtcttcctgtctgcccagcatgaacgcgaacatgtggaacacctggttaccacgtttgatcgtgtgctggaccgcctggcggatgaaaatctgctgagttggcagccgaccaacctgagtggtaatcaatcctaa

SEQ ID NO: 5 - DNA, 930 bp, Artificial Sequence
  EeaTA
atgaccacgaagaaagctgattacatttggttcaatggggagatggttcgctgggaagacgcgaaggtgcatgtgatgtcgcacgcgctgcactatggcacttcggtttttgaaggcatccgttgctacgactcgcacaaaggaccggttgtattccgccatcgtgagcatatgcagcgtctgcatgactccgccaaaatctatcgcttcccggtttcgcagagcattgatgagctgatggaagcttgtcgtgacgtgatccgcaaaaacaatctcaccagcgcctatatccgtccgctgatcttcgtcggtgatgttggcatgggagtaaacccgccagcgggatactcaaccgacgtgattatcgctgctttcccgtggggagcgtatctgggcgcagaagcgctggagcaggggatcgatgcgatggtttcctcctggaaccgcgcagcaccaaacaccatcccgacggcggcaaaagccggtggtaactacctctcttccctgctggtgggtagcgaagcgcgccgccacggttatcaggaaggtatcgcgctggatgtgaacggttatatctctgaaggcgcaggcgaaaacctgtttgaagtgaaagatggtgtgctgttcaccccaccgttcacctcctccgcgctgccgggtattacccgtgatgccatcatcaaactggcgaaagagctgggaattgaagtacgtgagcaggtgctgtcgcgcgaatccctgtacctggcggatgaagtgtttatgtccggtacggcggcagaaatcacgccagtgcgcagcgtagacggtattcaggttggcgaaggccgttgtggcccggttaccaaacgcattcagcaagccttcttcggcctcttcactggcgaaaccgaagataaatggggctggttagatcaagttaatcaataa

SEQ ID NO: 6 - DNA, 1362 bp, Artificial Sequence
  VfTA
atgaataagccacagtcatgggaagcgcgcgccgagacttattcgctttacggatttactgatatgccgagcctgcaccaacggggtacggtggtcgttacccatggcgaaggcccgtacatcgtggacgtgaacggccggcgttatttagatgcgaactctgggctgtggaatatggttgccggctttgaccacaaaggacttatcgatgcggcgaaagcacaatacgagcgatttcctggttatcacgcgttcttcgggcgaatgtcggatcagactgtgatgttaagcgaaaaactggtggaggtcagcccatttgatagtgggcgggtgttctataccaatagtggtagtgaagctaatgatacgatggtgaagatgctgtggttccttcatgctgcagaaggcaagccgcaaaaacgtaaaatcttgacccgctggaacgcttatcacggagtcaccgctgtctctgcctcaatgaccggcaaaccgtataactcggtttttgggctaccgctgccaggttttgtgcacctgacctgccctcattactggcgctatggcgaagaaggcgaaaccgaagaacagttcgtggcgcgcctggcccgtgaactggaagaaacaattcagcgcgaaggtgcagataccatcgctggtttcttcgcggagcctgtgatgggagccggcggtgtgattccgcccgccaaaggttacttccaggcgattttgccgatcctgcgtaagtatgacattccggttatcagcgacgaggtcatttgcggttttggtcgcacgggtaacacctggggttgcgttacttatgatttcacccccgatgcaatcatatccagtaaaaatttaacagctgggtttttcccgatgggcgcggtcattctgggcccggagctgtcaaaacgcctcgaaacagcgatcgaagcaattgaggaatttcctcatggcttcacggcgtctgggcatcctgttggctgtgcaatcgccttgaaagccattgacgttgtaatgaacgaaggcctggcagaaaacgtacgtcgcctggcgccgcgttttgaggaacgcctcaaacatattgcagaaaggccgaacattggcgagtacagaggcattggatttatgtgggcgctggaagcagtaaaagacaaagcctccaaaacgccatttgatgggaatctcagcgtcagcgagcgtatagccaatacgtgcacagatttaggtttgatttgtcgtccgcttggtcagtccgtagtgctgtgtcccccatttatcttaacggaggctcagatggatgaaatgtttgacaaactggaaaaggccctggataaagtttttgcggaggtagcgtaa

SEQ ID NO: 7 - DNA, 1380 bp, Artificial Sequence
  CvTA
atgcaaaaacaacgcaccacctcacaatggcgcgaactggatgccgcacaccacctgcacccgtttaccgacaccgcaagcctgaatcaggccggcgcccgtgttatgacccgcggcgaaggtgtgtatctgtgggattctgagggtaacaaaattatcgacggcatggctggtctgtggtgcgttaatgtcggctatggtcgtaaagattttgccgaagcggcccgtcgccaaatggaagaactgccgttctacaacacctttttcaaaaccacgcatccggcggtggttgaactgagcagcctgctggcggaagttacgccggccggctttgatcgtgtgttctataccaattcaggttcggaaagcgtggatacgatgatccgcatggttcgtcgctactgggacgtccagggcaaaccggaaaagaaaaccctgatcggtcgttggaacggctatcatggttctacgattggcggtgcaagtctgggcggtatgaaatacatgcacgaacagggcgatctgccgattccgggtatggcgcatatcgaacaaccgtggtggtacaaacacggcaaagatatgaccccggacgaatttggtgtcgtggcagctcgctggctggaagaaaaaattctggaaatcggcgccgataaagtggcggcctttgttggcgaaccgattcagggtgcgggcggtgtgattgttccgccggccacctattggccggaaattgaacgtatctgccgcaaatacgatgttctgctggtcgcagacgaagttatttgtggctttggtcgtaccggcgaatggttcggtcatcagcactttggcttccaaccggacctgtttacggcagctaaaggcctgagttccggttatctgccgatcggcgccgtcttcgtgggtaaacgcgttgcagaaggtctgattgctggcggtgattttaatcatggcttcacctatagcggtcacccggtctgtgcggccgtggcacatgctaatgtggcagctctgcgtgacgaaggcatcgtgcagcgcgttaaagatgacattggtccgtatatgcaaaaacgttggcgcgaaacgtttagccgtttcgaacacgtcgatgacgtgcgcggcgttggtatggtccaggcatttaccctggtgaaaaataaagctaaacgcgaactgtttccggatttcggcgaaattggtacgctgtgccgtgacatctttttccgcaacaatctgattatgcgtgcgtgtggtgatcacattgttagcgccccgccgctggttatgacccgcgcagaagtcgacgaaatgctggccgtggcggaacgctgcctggaagaatttgaacagaccctgaaagctcgtggcctggcgtaa

SEQ ID NO: 8 - DNA, 1362 bp, Artificial Sequence
  mVfTA
atgaataagccacagtcatgggaagcgcgcgccgagacttattcgctttacggatttactgatatgccgagcctgcaccaacggggtacggtggtcgttacccatggcgaaggcccgtacatcgtggacgtgaacggccggcgttatttagatgcgaactctgggctgtggaatatggttgccggctttgaccacaaaggacttatcgatgcggcgaaagcacaatacgagcgatttcctggttatcacgcgttcttcgggcgaatgtcggatcagactgtgatgttaagcgaaaaactggtggaggtcagcccatttgatagtgggcgggtgttctataccaatagtggtagtgaagctaatgatacgatggtgaagatgctgtggttccttcatgctgcagaaggcaagccgcaaaaacgtaaaatcttgacccgctggaacgcttttcacggagcgaccgctgtctctgcctcaatgaccggcaaaccgtataactcggtttttgggctaccgctgccaggttttgtgcacctgacctgccctcattactggcgctatggcgaagaaggcgaaaccgaagaacagttcgtggcgcgcctggcccgtgaactggaagaaacaattcagcgcgaaggtgcagataccatcgctggtttcttcgcggagcctgtgatgggagccggcggtgtgattccgcccgccaaaggttacttccaggcgattttgccgatcctgcgtaagtatgacattccggttatcagcgacgaggtcatttgcggttttggtcgcacgggtaacacctggggttgcgttacttatgatttcacccccgatgcaatcatatccagtaaaaatttaacagctgggtttttcccgatgggcgcggtcattctgggcccggagctgtcaaaacgcctcgaaacagcgatcgaagcaattgaggaatttcctcatggcttcacggcgtctgggcatcctgttggctgtgcaatcgccttgaaagccattgacgttgtaatgaacgaaggcctggcagaaaacgtacgtcgcctggcgccgcgttttgaggaacgcctcaaacatattgcagaaaggccgaacattggcgagtacagaggcattggatttatgtgggcgctggaagcagtaaaagacaaagcctccaaaacgccatttgatgggaatctcagcgtcagcgagcgtatagccaatacgtgcacagatttaggtttgatttgtcgtccgcttggtcagtccgtagtgctgtgtcccccatttatcttaacggaggctcagatggatgaaatgtttgacaaactggaaaaggccctggataaagtttttgcggaggtagcgtaa

SEQ ID NO: 9 - DNA, 1425 bp, Artificial Sequence
  BmTA
atgagtttaacagtgcaaaaaataaactgggagcaagttaaagaatgggatcgtaaatacctgatgaggacgtttagtacccaaaatgagtatcaacctgtaccaattgaatcaacagaaggtgactatttgattatgcctgatggcacaagattattagatttcttcaatcaactttattgtgtcaatttaggtcagaaaaatcaaaaagttaatgctgcaatcaaggaggctttagaccgatatggttttgtatgggatacttatgcaactgattataaagccaaagcagcaaagataattattgaggatattttaggtgatgaagactggccaggaaaagttaggttcgtatcaacaggaagtgaagcagtggaaacggcactgaatatcgcgaggttatatacaaatcgcccactagtggttacacgagaacacgattatcatggttggactggtggagctgctactgttactcgattaaggtcatatcgaagtggtttagttggggaaaattcagaatctttttcagcacaaatacccgggtcatcatataatagtgctgttttgatggcgccatcccctaacatgtttcaggatagtgacggcaacctgctaaaagatgaaaacggggaattgttgagtgtaaagtatacacgtcgtatgatcgaaaactatggtccggaacaagtggcagcagtgataactgaagtatcgcaaggtgcaggctctgctatgcccccatatgaatacattccacaaatccgaaaaatgacaaaagaactaggtgtcctttggattaatgatgaagttcttactggctttgggcgaacagggaagtggtttggatatcagcactatggggtacagccagatataatcactatgggtaaaggactctccagttcctcactccctgctggcgctgtcttagttagtaaggaaattgcagcgtttatggataagcaccgatgggagtcagtatccacgtatgccggtcatccagttgcgatggctgcggtctgtgcaaatttagaagtgatgatggaagaaaactttgttgagcaagcgaaggacagcggcgagtatataaggagtaaacttgaacttctgcaagaaaagcataaaagtataggaaatttcgacggatatggccttttatggatagtagatattgtgaatgccaagactaagactccttacgtaaaattggaccggaactttacgcacgggatgaatccaaatcaaatcccaacacaaatcattatgaaaaaagcgctagaaaaaggagtgctgattggtggagtaatgcctaatacaatgagaattggcgcatctttgaatgttagtcgcggagacatcgataaagcaatggatgcactggattatgcacttgactatttagaaagtggagaatggcagtaa

SEQ ID NO: 10 - DNA, 1374 bp, Artificial Sequence
  MmTA
atgttgactaacgaccaattggatcgctttgaccgcgaaaacttcttccacccaagtacacatctggcacagcatgcacgcggcgagagtccttcgcgcatcgtaaagactgcgaagggtgtattcattgaggatcgtgacgggaacaaactgttagacggctttggtggcttatactgcgtaaacgtggggtacggtcaagaatcaattattgaggcaattgcggaacaagcaaaggaacttgcgtactaccacgcgtacgcgggtcatggtaccgaagcttcaattaatttggcgaaaatggttatcgaccgcgccccagatcacatgagtaaagtctactttgggttgagtggtagtgacgctaatgagaccaatatcaagcttatctggtactataataacatcttagggcgcccggaaaagaaaaaaatcatctctcgctggcgtggatatcatggaagtggcttaatgacaggttctcttacgggcttggcaggcttccagcgtaaattcgatctgccgctggagcgtgtgtttcataccaccgcgccgtattactaccgccgcaaagacctggctatgagtgaagcggactttgtagctcattgcgttgcggaattagaaatgcgcatcgagcgtgaaggagctgataccattgcggcgttcattggtgagccagttttgggtacaggagggatcgtgcctccgcccgagggctattggaaagcaatctcggcggtgcttgagaagcacgatattctgcttattgctgacgaggttgtcactgggttcgggcgcttgggatctatgttcggaagcgatcactacggcttaaaacctgatttgatcactattgcaaaggggttaacctcagcgtacgcacctttatcaggcacaattgtcagtgacaaggtatggaaagtgctggaacagggtacggatgaaaacggccctattggtcatggatggacttactccgcacatcccattggcgcggcagcgggagtagcaaacttgaagttaatcgacgaacttgggttggtcaaaaacgccgcggagactggagcttatctgcgtgctgctatgaaagacgctctgtctgatcaccctcacgtgggagatatccgtggggaagggatgttaatggctgttgaatttgttaaagatcgtgagtcacgcacattttacgatccgtcagagaaagtgggaccgaatttagccgcggcattgattagcgaaggagttatcgcacgtgcaatgccagagggggatatcctgggatacgcccccccactttgtctgacacgcgaagaggcagaccaaattgttgctgcaactaaaaaggccgttattgccgttttgggctagtaa

SEQ ID NO: 11 - DNA, 972 bp, Artificial Sequence
  NfTA
atggcaagtatggataaggtcttttccgggtatcacgcccgccaaaaacttttggagcgcagcgataatccattcagtaagggtatcgcgtacgtggaggggaagttagtcttgccttcagacgcacgtattccgcttttggacgaaggattcatgcatggcgatctgacttatgacgtaaccaccgtttgggatggccgtttttttcgcttagatgatcacatgcagcgcattttagagtcctgtgataaaatgcgcttgaagttcccattggcacccagtaccgtcaaaaatattctggcggaaatggtcgcgaaaagcgggatccgcgacgcgttcgtagaggtcatcgtgactcgtggtcttacaggagtacgtggaagtaaacccgaagacctgtataataataatatctaccttttagtactgccttatgtctgggtaatggcaccagaaaaccagcttctgggtggctccgccatcatcacgcgtaccgtacgccgtacccctccaggcgcgttcgaccctacaatcaaaaacttgcaatggggagatcttactaagggcttgtttgaggcaatggaccgtggtgcgacgtatccatttcttacggacggtgacacgaatttaacggagggatctggatttaatatcgtactggttaaaaacggtatcatctacactcccgaccgtggggtgttacgtggtatcacgcgtaagtccgtgatcgacgttgcccgcgcgaataatattgacattcgcttggaagtggttcctgtagagcaggtttaccatagtgacgaaatctttatgtgcacaacagcgggcggcatcatgcctatcacgcttctggacggtcagccggtcaacgatggtcaagtagggccaatcaccaaaaagatctgggatggttattgggagatgcactacaacccagcctactcctttcctgttgattatggcagtgggtaa

SEQ ID NO: 12 - DNA, 993 bp, Artificial Sequence
  ArRTA
atggcattcagtgctgatacatctgaaattgtctatacacatgatacaggattagactatattacttacagtgattacgagttggacccagcgaaccccttggcaggcggcgcggcctggattgaaggggctttcgtacctccgtctgaagcccgtatttcaatttttgaccagggttatctgcatagcgacgtgacgtatacggtctttcacgtctggaatggcaacgcattccgtttagatgaccacatcgaacgcctgttttcaaacgccgagagtatgcgcattattccgcccttaactcaggatgaggttaaggagattgccctggagcttgtcgcgaaaactgaattacgtgaggccttcgtaagtgtatccattacccgcggctacagtagcacgccaggcgaacgcgatattacaaagcatcgcccacaagtgtatatgtacgctgtcccttatcaatggattgtacccttcgaccgcatccgcgatggcgtccatgccatggttgcccagtcagtacgtcgtactccgcgcagtagtattgatccgcaggtgaagaacttccaatggggcgatttgattcgtgctgttcaggagacacacgaccgtggatttgaggcccctcttttgcttgacggggatggtttactggcagagggatctggatttaatgtggtagtaattaaggatggcgtcgttcgtagtcctgggcgtgcggcgttgcccggcattacgcgcaaaacggtgttagagatcgcagaaagcttaggacacgaagcgattctggcagacattacgttagccgaacttttggatgcagacgaagtattaggctgcacaactgccggtggtgtttggccgtttgtttcggtagatggcaatccgatctcagatggagtgcccggccccattactcagagcatcattcgccgctattgggagttgaatgtagaatcatcttcgttacttacgccggtccagtactga

SEQ ID NO: 13 - DNA, 978 bp, Artificial Sequence
  AtTA
atggcttcgatggacaaggttttcgcggggtacgcggctcgccaagcgattctggaaagcacggagaccacaaatccattcgcaaaagggatcgcctgggtggaaggcgaactggtcccgttggcagaggcgcgtatccctttacttgaccaaggttttatgcatagcgacttaacctatgacgtaccgtcggtatgggatggacgtttcttccgcttggacgatcacatcacccgtctggaagcctcttgtacaaaactgcgccttcgcttacccctgcctcgcgaccaggtcaaacagatcttggtcgaaatggtagcaaaaagcggtattcgtgacgcgttcgtcgaattaattgtcacgcgtgggttgaaaggagtacgcgggactcgcccggaagacattgtcaataacttgtatatgttcgtgcaaccctacgtttgggttatggagcccgatatgcaacgtgtaggggggtcggcggttgtcgcacgtaccgttcgtcgtgtgcccccaggagccatcgaccccaccgtcaaaaacttgcaatggggggatcttgtccgtggtatgttcgaggccgcggatcgcggtgcaacatatccttttttaacagatggcgacgcacatctgactgaaggtagcggttttaacatcgttctggtaaaagatggtgtactgtatacgcctgatcgcggtgtcctgcaaggtgtcacgcgtaaaagcgtaattaacgcggcagaagccttcggcatcgaggttcgtgtcgaatttgtacccgtagagttggcctaccgttgcgacgaaattttcatgtgtaccaccgcggggggtattatgcctattaccactttagacggaatgcccgttaatggtggacaaatcggtcctatcaccaagaagatctgggatgggtattgggcaatgcactacgacgcggcatactccttcgaaatcgattataacgagcgcaactga